SEQUENCE LISTING (72 sequences)

SEQ ID NO: 1 - protein, 23 aa, Artificial
  Synthetic Anti-Her2 VL FR1
DIQMTQSPSSLSASVGDRVTITC

SEQ ID NO: 2 - protein, 15 aa, Artificial
  Synthetic Anti-Her2 VL FR2
WYQQKPGKAPKLLIY

SEQ ID NO: 3 - protein, 32 aa, Artificial
  Synthetic Anti-Her2 VL FR3
GVPSRFSGSRSGTDFTLTISSLQPEDFATYYC

SEQ ID NO: 4 - protein, 10 aa, Artificial
  Synthetic Anti-Her2 VL FR4
FGQGTKVEIK

SEQ ID NO: 5 - protein, 23 aa, Artificial
  Synthetic Anti-Her2 VL FR1
DIQMTQSPSSLSASVGDRVTITC

SEQ ID NO: 6 - protein, 15 aa, Artificial
  Synthetic Anti-Her2 VL FR2
WYQQKPGKAPKLLIY

SEQ ID NO: 7 - protein, 32 aa, Artificial
  Synthetic Anti-Her2 VL FR3
GVPSRFSGSGSGTDFTLTISSLQPEDFATYYC

SEQ ID NO: 8 - protein, 10 aa, Artificial
  Synthetic Anti-Her2 VL FR4
FGQGTKVEIK

SEQ ID NO: 9 - protein, 23 aa, Artificial
  Synthetic Anti-IgE VL FR1
DIQLTQSPSSLSASVGDRVTITC

SEQ ID NO: 10 - protein, 15 aa, Artificial
  Synthetic anti-IgE VL FR2
WYQQKPGKAPKLLIY

SEQ ID NO: 11 - protein, 32 aa, Artificial; identical to SEQ ID NO: 7
  Synthetic Anti-IgE VL FR3

SEQ ID NO: 12 - protein, 10 aa, Artificial
  Synthetic Anti-IgE VL FR4
FGQGTKVEIK

SEQ ID NO: 13 - protein, 23 aa, Artificial
  Synthetic Anti-IgE VL FR1
DIQLTQSPSSLSASVGDRVTITC

SEQ ID NO: 14 - protein, 15 aa, Artificial
  Synthetic Anti-IgE VL FR2
WYQQKPGKAPKLLIY

SEQ ID NO: 15 - protein, 32 aa, Artificial; identical to SEQ ID NO: 7
  Synthetic Anti-IgE VL FR3

SEQ ID NO: 16 - protein, 10 aa, Artificial
  Synthetic Anti-IgE VL FR4
FGQGTKVEIK

SEQ ID NO: 17 - protein, 23 aa, Artificial
  Anti-VEGF VL FR1
DIQMTQSPSSLSASVGDRVTITC

SEQ ID NO: 18 - protein, 15 aa, Artificial
  Synthetic Anti-VEGF VL FR2
WYQQKPGKAPKVLIY

SEQ ID NO: 19 - protein, 32 aa, Artificial; identical to SEQ ID NO: 7
  Synthetic Anti-VEGF VL FR3

SEQ ID NO: 20 - protein, 10 aa, Artificial
  Synthetic Anti-VEGF VL FR4
FGQGTKVEIK

SEQ ID NO: 21 - protein, 23 aa, Artificial
  Synthetic Anti-CD11a VL FR1
DIQMTQSPSSLSASVGDRVTITC

SEQ ID NO: 22 - protein, 15 aa, Artificial
  Synthetic Anti-CD11a VL FR2
WYQQKPGKAPKLLIY

SEQ ID NO: 23 - protein, 32 aa, Artificial; identical to SEQ ID NO: 7
  Synthetic Anti-CD11a VL FR3

SEQ ID NO: 24 - protein, 10 aa, Artificial
  Synthetic Anti-CD11a VL FR4
FGQGTKVEIK

SEQ ID NO: 25 - protein, 23 aa, Artificial
  Synthetic Anti-CD20 VL FR1
DIQMTQSPSSLSASVGDRVTITC

SEQ ID NO: 26 - protein, 15 aa, Artificial
  Synthetic Anti-CD20 VL FR2
WYQQKPGKAPKLLIY

SEQ ID NO: 27 - protein, 32 aa, Artificial; identical to SEQ ID NO: 7
  Synthetic Anti-CD20 VL FR3

SEQ ID NO: 28 - protein, 10 aa, Artificial
  Synthetic Anti-CD20 VL FR4
FGQGTKVEIK

SEQ ID NO: 29 - protein, 23 aa, Artificial
  Synthetic Anti-CD20 VL FR1
QIVLSQSPAILSASPGEKVTMTC

SEQ ID NO: 30 - protein, 15 aa, Artificial
  Synthetic Anti-CD20 VL FR2
WFQQKPGSSPKPWIY

SEQ ID NO: 31 - protein, 32 aa, Artificial
  Synthetic Anti-CD20 VL FR3
GVPVRFSGSGSGTSYSLTISRVEAEDAATYYC

SEQ ID NO: 32 - protein, 10 aa, Artificial
  Synthetic Anti-CD20 VL FR4
FGGGTKLEIK

SEQ ID NO: 33 - protein, 23 aa, Artificial
  Synthetic Anti-BR3 VL FR1
DIQMTQSPSSLSASVGDRVTITC

SEQ ID NO: 34 - protein, 15 aa, Artificial
  Synthetic Anti-BR3 VL FR2
WYQQKPGKAPKLLIY

SEQ ID NO: 35 - protein, 32 aa, Artificial; identical to SEQ ID NO: 7
  Synthetic Anti-BR3 VL FR3

SEQ ID NO: 36 - protein, 10 aa, Artificial
  Synthetic Anti-BR3 VL FR4
FGQGTKVEIK

SEQ ID NO: 37 - protein, 30 aa, Artificial
  Synthetic Anti-Her2 VH FR1
EVQLVESGGGLVQPGGSLRLSCAASGFNIK

SEQ ID NO: 38 - protein, 14 aa, Artificial
  Synthetic Anti-Her2 VH FR2
WVRQAPGKGLEWVA

SEQ ID NO: 39 - protein, 32 aa, Artificial
  Synthetic Anti-Her2 VH FR3
RFTISADTSKNTAYLQMNSLRAEDTAVYYCSR

SEQ ID NO: 40 - protein, 11 aa, Artificial
  Synthetic Anti-Her2 VH FR4
WGQGTLVTVSS

SEQ ID NO: 41 - protein, 30 aa, Artificial
  Synthetic Anti-Her2 VH FR1
EVQLVESGGGLVQPGGSLRLSCAASGFTFT

SEQ ID NO: 42 - protein, 14 aa, Artificial
  Synthetic Anti-Her2 VH FR2
WVRQAPGKGLEWVA

SEQ ID NO: 43 - protein, 32 aa, Artificial
  Synthetic Anti-Her2 VH FR3
RFTLSVDRSKNTLYLQMNSLRAEDTAVYYCAR

SEQ ID NO: 44 - protein, 11 aa, Artificial
  Synthetic Anti-Her2 VH FR4
WGQGTLVTVSS

SEQ ID NO: 45 - protein, 31 aa, Artificial
  Synthetic Anti-IgE VH FR1
EVQLVESGGGLVQPGGSLRLSCAVSGYSITS

SEQ ID NO: 46 - protein, 14 aa, Artificial
  Synthetic Anti-IgE VH FR2
WIRQAPGKGLEWVA

SEQ ID NO: 47 - protein, 32 aa, Artificial
  Synthetic Anti-IgE VH FR3
RITISRDDSKNTFYLQMNSLRAEDTAVYYCAR

SEQ ID NO: 48 - protein, 11 aa, Artificial
  Synthetic Anti-IgE VH FR4
WGQGTLVTVSS

SEQ ID NO: 49 - protein, 31 aa, Artificial; identical to SEQ ID NO: 45
  Synthetic Anti-IgE VH FR1

SEQ ID NO: 50 - protein, 14 aa, Artificial
  Synthetic Anti-IgE VH FR2
WIRQAPGKGLEWVA

SEQ ID NO: 51 - protein, 32 aa, Artificial; identical to SEQ ID NO: 47
  Synthetic Anti-IgE VH FR3

SEQ ID NO: 52 - protein, 11 aa, Artificial
  Synthetic Anti-IgE VH FR4
WGQGTLVTVSS

SEQ ID NO: 53 - protein, 30 aa, Artificial
  Synthetic Anti-VEGF VH FR1
EVQLVESGGGLVQPGGSLRLSCAASGYTFT

SEQ ID NO: 54 - protein, 14 aa, Artificial
  Synthetic Anti-VEGF VH FR2
WVRQAPGKGLEWVG

SEQ ID NO: 55 - protein, 32 aa, Artificial
  Synthetic Anti-VEGF VH FR3
RFTFSLDTSKSTAYLQMNSLRAEDTAVYYCAK

SEQ ID NO: 56 - protein, 11 aa, Artificial
  Synthetic Anti-VEGF VH FR4
WGQGTLVTVSS

SEQ ID NO: 57 - protein, 30 aa, Artificial
  Synthetic Anti-CD11a VH FR1
EVQLVESGGGLVQPGGSLRLSCAASGYSFT

SEQ ID NO: 58 - protein, 14 aa, Artificial
  Synthetic Anti-CD11a VH FR2
WVRQAPGKGLEWVG

SEQ ID NO: 59 - protein, 32 aa, Artificial
  Synthetic Anti-CD11a VH FR3
RFTISVDKSKNTLYLQMNSLRAEDTAVYYCAR

SEQ ID NO: 60 - protein, 11 aa, Artificial
  Synthetic Anti-CD11a VH FR4
WGQGTLVTVSS

SEQ ID NO: 61 - protein, 30 aa, Artificial; identical to SEQ ID NO: 53
  Synthetic Anti-CD20 VH FR1

SEQ ID NO: 62 - protein, 14 aa, Artificial
  Synthetic Anti-CD20 VH FR2
WVRQAPGKGLEWVG

SEQ ID NO: 63 - protein, 32 aa, Artificial; identical to SEQ ID NO: 59
  Synthetic Anti-CD20 VH FR3

SEQ ID NO: 64 - protein, 11 aa, Artificial
  Synthetic Anti-CD20 VH FR4
WGQGTLVTVSS

SEQ ID NO: 65 - protein, 30 aa, Artificial
  Synthetic Anti-CD20 VH FR1
QVQLQQPGAELVKAGASVKMSCKASGYTFT

SEQ ID NO: 66 - protein, 14 aa, Artificial
  Synthetic Anti-CD20 VH FR2
WVKQTPGRGLEWIG

SEQ ID NO: 67 - protein, 32 aa, Artificial
  Synethetic Anti-CD20 VH FR3
KATLTADCSSSTAYMQLSSLTSEDSAVYYCAR

SEQ ID NO: 68 - protein, 11 aa, Artificial
  Synthetic Anti-CD20 VH FR4
WGAGTTVTVSA

SEQ ID NO: 69 - protein, 30 aa, Artificial
  Synthetic Anti-BR3 VH FR1
EVQLVESGGGLVQPGGSLRLSCAASGFTVT

SEQ ID NO: 70 - protein, 14 aa, Artificial
  Synthetic Anti-BR3 VH FR2
WVRQAPGKGLEWVG

SEQ ID NO: 71 - protein, 32 aa, Artificial
  Synthetic Anti-BR3 VH FR3
RFTISRDTSKNTFYLQMNSLRAEDTAVYYCAQ

SEQ ID NO: 72 - protein, 11 aa, Artificial
  Synthetic Anti-BR3 VH FR4
WGQGTLVTVSS

(19) United States
(12) Patent Application Publication
(10) Pub. No.: US 2007/0161089 A1
(43) Pub. Date: Jul. 12, 2007

(54) Method of Producing Pan-Specific Antibodies

(75) Inventors: Jihong Yang, Foster City, CA (US); Anan Chuntharapai, Colma, CA (US)

Correspondence Address: MERCHANT & GOULD PC, P.O. BOX 2903, MINNEAPOLIS, MN, 55402-0903, US

(73) Assignee: GENENTECH, INC., South San Francisco, CA (US)

(21) Appl. No.: 11/556,917

(22) Filed: Nov. 6, 2006

Related U.S. Application Data
(60) Provisional application No. 60/735,258, filed on Nov. 8, 2005.

(51) Int. Cl. C12P 21/08; C07K 16/42

(52) U.S. Cl. 435/70.21; 530/388.15

(57) ABSTRACT

Pan-specific anti-humanized antibody antibody (anti-hzAbAb) are described as well as and methods for generating and using pan-specific anti-humanized antibody antibodies (anti-hzAbAb) that selectively bind related antibodies such as those therapeutic humanized having variable domain framework regions sharing at least 80% sequence identity and/or those having variable domain framework regions sharing at least 80% sequence identity with a variable domain consensus sequence.

### BACKGROUND OF THE INVENTION

Therapeutic antibodies are useful agents in clinical use today. Examples include Avastin™ (Genentech, Inc.), Zenapax® (Protein Design Labs), Zevalin® (Biogen IDEC, Inc.), Campath® (Genzyme Oncology), and the like. In general, the variable domains of humanized antibodies contain human framework regions (FRs) and nonhuman hypervariable regions (CDRs or HVLs). Humanized antibodies thus provide efficient antigen binding affinities while exposing patients to minimal nonhuman protein sequences. A variety of humanized antibodies directed against different antigens can be efficiently developed and manufactured, for example, using identical or related variable domain framework regions and substituting hypervariable regions (CDRs or HVLs) specific to desired antigens. See, for example, U.S. Pat. No. 6,054,297 to Carter et al., describing production of humanized antibody variable domains. This patent describes use of human heavy and light chain variable domain consensus sequences substituted with a set of hypervariable domains (CDRs and/or HVLs) derived from a nonhuman antibody. The humanized antibody can contain further amino acid substitutions, for example, in the framework regions, as described therein.

Reagents that bind therapeutic antibodies are needed for use in preclinical and clinical pharmacokinetic and pharmacodynamic assays, as well as for quality control studies. A specific reagent, such as an antibody that specifically binds the therapeutic antibody, is generally utilized for such assays. Because many different therapeutic humanized antibodies can be produced using identical or related variable domain framework regions, a pan-specific reagent capable of selectively binding a number of different humanized antibodies would streamline assay procedures and reduce time and costs required to produce reagents that indd only a single therapeutic antibody.

Thus, there is a need for pan-specific reagents, such as pan-specific antibodies, capable of selectively binding a group of therapeutic antibodies such as a group of related humanized antibodies having the same or related variable domain framework regions.

### SUMMARY OF THE INVENTION

The present invention provides pan-specific reagents and methods for the production and use of pan-specific reagents that selectively bind related antibodies having the same or similar variable domain framework regions. The pan-specific reagent can be an antibody such as a monoclonal antibody and including antigen-binding fragments of antibodies. For example, pan-specific anti-humanized antibody antibodies (anti-hzAbAb) described herein selectively bind two or more humanized antibodies in a group of related antibodies having variable domain framework regions sharing at least 80% amino acid sequence identity, and may at least share about 85%, 90%, or 95% identity. In another example, the related heavy chain and/or light chain variable domain framework regions share at least 80% amino acid sequence identity with the framework regions of a variable domain consensus sequence. The consensus sequence can be derived from a Kabat compilation of variable domain sequences, for example. The framework sequence can share at least 85%, at least 90%, or at least 95% sequence identity to the framework regions of a consensus sequence variable domain, for example.

In one embodiment, the pan-specific anti-hzAbAb selectively binds two or more related humanized antibodies that bind different antigens where the variable domain framework regions are identical or have about 1 to about 10 amino acid substitutions. The framework regions can have, for example, 1, 2, 3, 4, 5, 6, 7, 8, 9 or about 10 amino acid substitutions in some embodiments. The related humanized antibodies can comprise variable domain framework regions having identical amino acid sequences to framework regions of a consensus heavy chain or light chain variable domain, or both, or may contain about 1 to about 10 amino acid substitutions in the consensus framework regions. The framework regions can have, for example, 1, 2, 3, 4, 5, 6, 7, 8, 9, or 10 amino acid substitutions. The consensus sequence variable domain framework region, for example, can contain at each position the most abundant amino acid at that position as reported in a compilation of antibody variable domain sequences, such as those described by Kabat et al. (5th (1991), 4th (1987), and 3rd (1983) editions of Sequences of Proteins of Immunological Interest, Public Health Service, National Institutes of Health, Bethesda, Md.).

In another embodiment, a pan-specific anti-hzAbAb selectively binds at least two related humanized antibodies having variable domain framework regions derived from a variable domain consensus sequence obtained from a Kabat compilation of Kappa Subgroup III heavy chain variable domains and Kappa Subgroup I light chain variable domains. In a particular embodiment, the pan-specific antibody selectively binds at least two of humanized anti-Her2, humanized anti-VEGF, humanized anti-IgE, humanized anti-CD11a, humanized anti-CD20, and humanized anti-BR3 antibodies.

The present invention also includes a method for producing a pan-specific antibody that selectively binds related humanized antibodies and does not bind antibodies outside the related group, for example, those antibodies having related variable domain framework regions that share at least 80% sequence identity, and/or have at least 80% sequence identity with framework regions of a consensus sequence variable domain. The framework regions may share at least 85%, 90%, or 95% sequence identity, for example one or more of the framework regions shown in FIGS. 1 and 2.

In a specific embodiment, the invention includes a process for producing a pan-specific antibody that selectively binds at least two humanized antibodies from a group of related humanized antibodies, comprising the steps of: (a) immunizing an animal with at least two humanized antibodies having related variable domain framework regions that share at least 80% sequence identity and/or have at least 80% identity with the framework region or a variable domain consensus sequence; and (b) harvesting an antibody that selectively binds said at least two related humanized antibodies. In an embodiment, the animal is immunized with two or more of humanized anti-Her2, humanized anti-VEGF, humanized anti-IgE, humanized anti-CD 11a, humanized anti-CD20, and humanized anti-BR3, and may be immunized with 3, 4, 5, 6, 7, 8, 9, or 10 of such antibodies.

In another embodiment, the invention includes assay methods using a pan-specific anti-hzAbAb to detect a humanized antibody in a sample. The present invention also includes articles of manufacture comprising a container, a composition within the container comprising a pan-specific anti-hzAbAb, and a package insert containing instructions for the use of the composition. Such articles of manufacture can be used in preclinical and clinical assays.

### BRIEF DESCRIPTION OF THE FIGURES

FIG. 1 is a diagram of an antibody showing the structure and relationship of heavy and light chain variable and constant domains, and showing hypervariable regions (HVLs and/or CDRs).

FIG. 2 is a listing of amino acid sequences contained in the light chain variable domain framework regions of related humanized antibodies and control antibodies. Boxed regions indicate locations of complementarity-determining regions as defined by Kabat et al., Sequences of Proteins of Immunological Interest, 4th Ed. Public Health Service, National Institutes of Health, Bethesda, Md. (1987). The four framework (FR) regions flanking the three CDRs are indicated.

FIG. 3 is a listing of amino acid sequences contained in the heavy chain variable domain framework regions of related humanized antibodies and control antibodies. Boxed regions indicate locations of complementarity-determining regions as defined by Kabat et al., Sequences of Proteins of Immunological Interest, 4th Ed. Public Health Service, National Institutes of Health, Bethesda, Md. (1987). The four framework (FR) regions flanking the three CDRs are indicated.

FIG. 4 is a standard curve correlating the concentration (nM) of purified cynomolgus monkey anti-VEGF antibody polyclonal antibodies with response units (RU) in an ECLA assay.

FIG. 5 is a graph showing selective binding of a pan-specific anti-humanized antibody antibody (anti-hzAbAb) produced from hybridoma clone 2H9.3.2 to related humanized antibodies.

FIG. 6 is a graph showing selective binding of a pan-specific anti-humanized antibody antibody (anti-hzAbAb) produced from hybridoma clone 10C4.1 to related humanized antibodies.

### DETAILED DESCRIPTION OF THE EMBODIMENTS

A. Definitions

The following terms, as used herein, are intended to have the following definitions:

The term “amino acid” is used in its broadest sense and is meant to include the naturally occurring L α-amino acids or residues. The commonly used one and three letter abbreviations for naturally occurring amino acids are used herein (Lehninger, 1975, Biochemistry, 2d ed., pp. 71-92, Worth Publishers, New York). The term includes all D-amino acids as well as chemically modified amino acids such as amino acid analogs, naturally occurring amino acids that are not usually incorporated into proteins such as norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio, In: The Peptides: Analysis, Synthesis, Biology, Gross and Meiehofer, Eds., Vol. 5 p 341, Academic Press, Inc, N.Y. 1983, which is incorporated herein by reference.

The term “antigen cocktail” refers to a mixture of peptides or polypeptides used to stimulate the production of antibodies. The term “mixture” refers to the combination of at least 2 or more molecules. An antigen cocktail contains at least 2 or more molecules that are immunogens. In the examples below, a mixture of seven humanized therapeutic antibodies having related framework regions was used as an antigen cocktail to stimulate production of anti-humanized antibody antibodies.

The term “antibody” is used in the broadest sense and specifically includes monoclonal antibodies (including full length monoclonal antibodies), multispecific antibodies (e.g., bispecific antibodies), and antibody fragments that exhibit a desired biological activity or function.

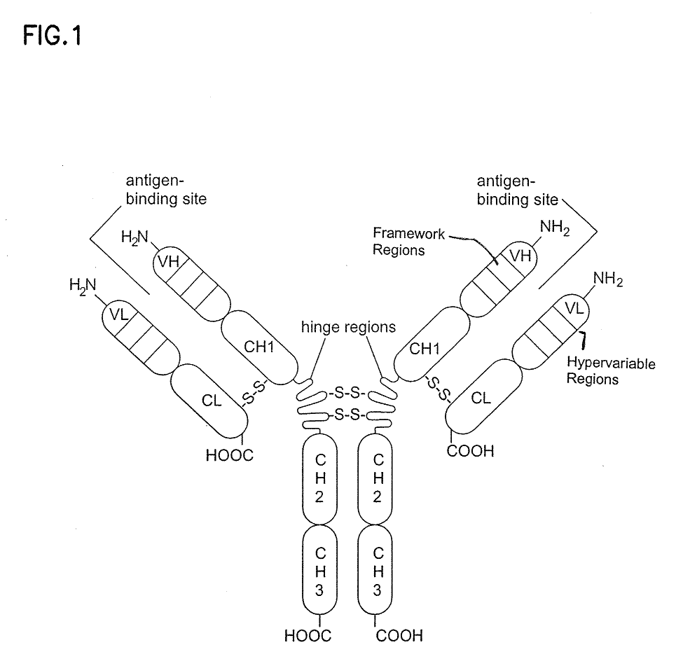

As shown in FIG. 1, an antibody generally comprises two heavy chains and two light chains, each comprising a variable domain and a constant domain. Each variable domain contains a hypervariable region containing three CDRs or HVLs flanked by four segments of the framework region. As used herein, the “framework region” contains all four segments FR1, FR2, FR3, and FR4 that flank a set of three hypervariable regions (CDRs or HVLs).

Antibodies can be chimeric, humanized, or human, for example, and can be antigen-binding fragments of these. Antibodies are generally produced by immunizing an animal with an antigen, and can be produced by recombinant technology, or by synthesis of the amino acid sequence, for example. “Antibody fragments” comprise a portion of a full-length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies such as bispecific antibodies, for example formed from antibody fragments. “Functional fragments” substantially retain binding to an antigen of the full-length antibody, and retain a biological activity.

The term “monoclonal antibody” as used herein refers to an antibody obtained from a population of substantially homogeneous antibodies, i.e., the individual antibodies of the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, monoclonal antibodies may be made by the hybridoma method first described by Kohler et al., 1975, Nature 256:495, or may be made by recombinant DNA methods (see, e.g., U.S. Pat. No. 4,816,567). The “monoclonal antibodies” may also be isolated from phage antibody libraries using the techniques described in Clackson et al., 1991, Nature 352:624-628 and Marks et al., 1991, J. Mol. Biol. 222:581-597, for example.

“Chimeric” antibodies (immunoglobulins) contain a portion of a heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., 1984, Proc. Natl. Acad. Sci. USA 81:6851-6855). A “humanized antibody”, as the term is used herein, is a subset of chimeric antibodies.

“Humanized” forms of nonhuman (e.g., murine) antibodies are chimeric antibodies that contain minimal sequence derived from nonhuman immunoglobulin. For the most part, humanized antibodies are human immunoglobulins (recipient or acceptor antibody) in which variable domain hypervariable region residues of the recipient antibody are replaced by hypervariable region residues from a nonhuman species (donor antibody), such as mouse, rat, rabbit, or nonhuman primate having the desired specificity, affinity, and capacity. The hypervariable regions can be complementarity-determining regions (CDRs) defined by sequence (see, for example Kabat 1991, 1987, 1983), or hypervariable loops (HVLs) defined by structure (see for example, Chothia 1987), or both. In some embodiments, the variable domain framework regions are derived from a consensus sequence variable domain, for example, containing at each residue an amino acid compiled as most abundant at that position in a class or subclass of human immunoglobulin variable domains, for example, in a Kabat compilation. In some instances, one or more amino acids of the variable domain framework region (FR) of the human immunoglobulin or consensus sequence is replaced with one or more corresponding residues of the nonhuman donor antibody and/or one or more amino acids of the donor antibody hypervariable regions is replaced with one or more corresponding human residues of the human recipient variable domain. In some instances, one or more residues of the variable domain framework regions and/or hypervariable regions is a residue not found at the corresponding position in the recipient antibody or in the donor antibody. Modifications to the amino acid sequence of the variable domain framework regions and hypervariable regions are generally made to further refine antibody performance, for example, improve binding affinity. In general, the humanized antibodies used to produce the pan-specific antibodies described herein will comprise substantially all of at least one, and typically two, variable domains, in which all or substantially all of the hypervariable region residues (CDRS or HVLs) correspond to those of a nonhuman immunoglobulin and all or substantially all of the framework region (FR) residues correspond to those of a human variable domain consensus sequence, and may include one or more amino acid substitutions. In some embodiments, the number of amino acid substitutions in the human consensus framework region is typically no more than 10, and may be, for example, 1, 2, 3, 4, 5, 6, 7, 8, 9, or 10 substitutions in the heavy chain variable domain framework regions, and 0, 1, 2, 3, 4, 5, 6, 7, 8, 9, or 10 substitutions in the light chain variable domain framework region. The humanized antibody optionally comprises at least a portion of an immunoglobulin constant region, typically that of a human immunoglobulin (see FIG. 1). For further details, see Jones et al., 1986, Nature 321:522-525; Reichmann et al., 1988, Nature 332:323-329; and Presta, 1992, Curr. Op. Struct. Biol. 2:593-596.

An “Fv” fragment is an antibody fragment that contains a complete antigen recognition and binding site, and generally comprises a dimer of one heavy and one light chain variable domain in tight association that can be covalent in nature, for example in a single chain variable domain fragment (scFv). It is in this configuration that the three hypervariable regions of each variable domain interact to define an antigen-binding site on the surface of the V_{H}-V_{L} dimer. Collectively, the six hypervariable regions or a subset thereof confer antigen binding specificity to the antibody. However, even a single variable domain comprising only three hypervariable regions specific for an antigen has the ability to recognize and bind antigen.

“Single-chain Fv” or “scFv” antibody fragments comprise the V_{H} and V_{L} domains of antibody, where these domains are present in a single polypeptide chain. Generally, the Fv polypeptide further comprises a polypeptide linker between the V_{H} and V_{L} domains that enables the scFv to form the desired structure for antigen binding. For a review of scFv, see Pluckthun, 1994, In: The Pharmacology of Monoclonal Antibodies, Vol. 113, Rosenburg and Moore eds. Springer-Verlag, New York, pp. 269-315.

A “Fab” fragment includes a variable domain and a constant domain of the light chain and a variable domain and the first constant domain (C_{H}1) of the heavy chain. A Fab′ fragment includes one or more cysteine carboxy terminal linkages to the heavy or light chains. F(ab′)₂ antibody fragments comprise a pair of Fab fragments that are generally covalently linked near their carboxy termini by hinge cysteines. Other chemical couplings of antibody fragments are also known.

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two variable domains on the same chain, the variable domains are forced to pair with complementary domains of another chain, creating two antigen-binding sites. Diabodies are described more fully, for example, in EP 404,097; WO 93/11161; and Hollinger et al., 1993, Proc. Natl. Acad. Sci. USA, 90:6444-6448.

The expression “linear antibodies” refers to antibodies as described in Zapata et al., 1995, Protein Eng., 8(10):1057-1062. Briefly, these antibodies contain a pair of tandem Fd segments (V_{H}-C_{H}1-V_{H}-C_{H}1) that, together with complementary light chain polypeptides, form a pair of antigen binding regions. Linear antibodies can be bispecific or monospecific.

The term “monobody” as used herein, refers to an antigen binding molecule with a heavy chain variable domain and no light chain variable domain. A monobody can bind to an antigen in the absence of light chains and typically has three hypervariable regions, for example CDRs designated CDRH1, CDRH2, and CDRH3. A heavy chain IgG monobody has two heavy chain antigen binding molecules connected by a disulfide bond. The heavy chain variable domain comprises one or more hypervariable regions, preferably a CDRH3 or HVL-H3 region.

The term “hypervariable region” when used herein refers to the amino acid residues of an antibody that are responsible for antigen-binding. The hypervariable region comprises amino acid residues from a “complementarity-determining region” or “CDR” (defined by sequence as residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (defined by structure and differing for each antibody; see, for example: Chothia and Lesk, 1987, J. Mol. Biol. 196:901-917). In one example, HVL residues can include, 26-32 (L1), 50-52 (L2) and 91-96 (L3) in the light chain variable domain and 26-32 (H1), 53-55 (H2) and 96-101 (H3) in the heavy chain variable domain.

“Framework region” or “FR” residues are those variable domain residues flanking the hypervariable region residues as herein defined. In general, a variable domain contains three hypervariable regions flanked by four sequences of the framework region (FR1, FR2, FR3, and FR4).

The term “consensus sequence”, as used herein, refers to an artificial variable domain sequence comprising at each position the residue that is most abundant at that position in the variable domains of a group of antibodies of a particular class, for example, as described in the compilations of Kabat et al., Sequences of Proteins of Immunological Interest,. Public Health Service, National Institutes of Health, Bethesda, Md. (5th Ed, 1991); (4th Ed 1987); (3rd Ed 1983). The consensus variable domain sequences do not have any known antibody binding specificity or affinity.

The term “detecting” is used in the broadest sense to include both qualitative and quantitative measurements of a specific molecule, for example, measurements of a specific analyte molecule such as a pan-specific anti-hzAbAb. In one aspect, a detection method as described herein is used to identify the mere presence of an analyte molecule of interest in a sample. In another aspect, the detection method can be used to quantify an amount of analyte molecule in a sample. In still another aspect, the method can be used to determine the relative binding affinity of an analyte molecule of interest for a target molecule.

The term “detecting agent” refers to an agent that detects an analyte molecule, either directly via a label, such as a fluorescent, enzymatic, radioactive, or chemiluminescent label, that can be linked to the detecting agent, or indirectly via a labeled binding partner, such as an antibody or receptor that specifically binds the detecting agent. Examples of detecting agents include, but are not limited to, an antibody, antibody fragment, soluble receptor, receptor fragment, and the like. In an embodiment, the detecting agent can be expressed on a phage.

The term “label” includes agents that amplify a signal produced by a detecting agent. The label can be a radiologic, photoluminescent, chemiluminescent, or electrochemiluminescent chemical moiety, an enzyme that converts a colorless substrate into a colored product, and the like.

The term “capture reagent” refers to a reagent capable of binding and capturing a target molecule or analyte molecule in a sample. Typically, a capture reagent is immobilized, for example, on a solid substrate, such as a microparticle or bead, microtiter plate, column resin, and the like. The capture reagent can be an antigen, soluble receptor, antibody, a mixture of different antibodies, and the like.

The term “target molecule” refers to a specific binding target of an analyte molecule, for example identified by electrochemiluminescence assay (ECLA), defined infra. A target molecule is typically a small molecule, polypeptide, or polypeptide fragment. The target molecule can be, for example, an antigen if the analyte molecule is an antibody, a receptor or antibody if the analyte molecule is a small molecule or polypeptide, a polypeptide or small molecule if the analyte molecule is a soluble receptor, a phage expressing antibody, soluble receptor, or fragments thereof if the analyte molecule is a polypeptide or small molecule. The target molecule can be, for example, a polypeptide such as a humanized antibody having therapeutic activity. In one embodiment, the target molecule is a humanized antibody and the analyte molecule is a pan-specific anti-hzAbAb that selectively binds the humanized therapeutic antibody.

“Analyte” and “analyte molecule,” as used herein, refer to a molecule that is analyzed, for example by ECLA, and includes, but is not limited to, small molecules, polypeptides, polypeptide fragments, antibodies, antibody fragments, phage, displayed polypeptides, and the like. An analyte molecule has a binding affinity for the target molecule.

“Polypeptide” refers to a peptide or protein containing two or more amino acids linked by peptide bonds, and includes peptides, oligimers, proteins, and the like. Polypeptides can contain natural, modified, or synthetic amino acids. Polypeptides can also be modified naturally, such as by post-translational processing, or chemically, such as amidation acylation, cross-linking, and the like.

As used herein, an “anti-humanized antibody antibody (anti-hzAbAb)” is an antibody that binds a humanized antibody. For example, an anti-HuMAb4D5 antibody is an antibody that binds HuMAb4D5, a therapeutic humanized antibody that binds the antigen Her2.

“Low affinity”, as used herein, means an analyte molecule having a dissociation rate constant (K_{dissoc}) generally greater then 10⁻⁶ l/sec for a target molecule. Preferably the K_{dissoc} of the analyte molecule for the target molecule is 10⁻⁵ l/sec or greater, 10⁻⁴1/sec or greater, 10⁻³1/sec or greater, or 10⁻² l/sec or greater. Useful low affinity antibodies typically have a dissociation rate constant of about 10⁻³ to 10⁻⁵ l/sec. A molecule with a high dissociation rate constant (K_{dissoc}) is likely to have low affinity, as the equilibrium dissociation constant, K_{D}=K_{dissoc}/K_{assoc}. A molecule with an equilibrium constant (K_{D}) equal to or greater than about 10⁻⁸ M has low binding affinity. Useful low affinity antibodies can have a K_{D} of about 10⁻⁶ M to about 10⁻⁸ M, for example.

Electrochemiluminescence assay or “ECLA” is an electrochemical assay in which bound analyte molecule is detected by a label linked to a detecting agent (target molecule). An electrode electrochemically initiates luminescence of a chemical label linked to a detecting agent. Light emitted by the label is measured by a photodetector and indicates the presence or quantity of bound analyte molecule/target molecule complexes. ECLA methods are described, for example, in U.S. Pat. Nos. 5,543,112; 5,935,779; and 6,316,607. Signal modulation can be maximized for different analyte molecule concentrations for precise and sensitive measurements.

The term “related,” as used herein when referring to polypeptides, including antibodies, refers to a group that shares amino acid sequence identity, for example at least 80% identity when compared to a reference sequence. For example, “related humanized antibodies” contain heavy and/or light chain variable domains comprising framework regions sharing about 80% to 100% sequence identity. For example, a group of related humanized antibodies may contain identical variable domain framework regions (heavy chain, light chain, or both) and distinct hypervariable regions (CDRs or HVLs). In another example, a group of related humanized antibodies contain variable domain framework regions sharing at least 80% sequence identity, and may share at least 85%, 90%, or 95% sequence identity, for example. In one example, related humanized antibodies contain variable domain framework regions derived from a human variable domain consensus sequence. A variable domain framework sequence that is “derived from” a variable domain consensus sequence may contain at each framework residue the corresponding residue of the consensus sequence or may contain the consensus sequence with one or more amino acid substitution. The “derived” framework region may contain, for example, 1, 2, 3, 4, 5, 6, 7, 8, 9, or 10 amino acid substitutions in the consensus sequence framework regions.

Antibody “binding affinity” may be determined, for example, by equilibrium methods such as enzyme-linked immunoabsorbent assay (ELISA) or radioimmunoassay (RIA), ECLA, or by kinetics, for example using BIACORE™ analysis.

“Mammal” for purposes of treatment or therapy refers to any animal classified as a mammal, including humans, domestic and farm animals, and zoo, sports, or pet animals, such as dogs, horses, cats, cows, and the like. Preferably, the mammal is human.

A nucleic acid sequence or polynucleotide is “operably linked” when it is placed into a functional relationship with another nucleic acid sequence. Generally, “operably linked” means that the DNA sequences being linked are contiguous. Linking is accomplished by ligation at convenient restriction sites. If such sites do not exist, synthetic oligonucleotide adaptors or linkers are used in accordance with conventional practice.

The term “pan-specific antibody” as used herein refers to an antibody that selectively binds at least two members of a group of related polypeptides and does not selectively bind polypeptides outside the group of related polypeptides. In a particular embodiment, a pan-specific antibody described herein selectively binds a group of humanized antibodies having related variable domain framework regions that share at least 80% sequence identity, and/or binds a group of humanized antibodies having related variable domain framework regions sharing at least 80% sequence identity with the framework regions of a human consensus sequence variable domain.

“Percent (%) amino acid sequence identity” with respect to a polypeptide is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full length of the sequences being compared. The ALIGN-2 program is publicly available through Genentech, Inc., South San Francisco, Calif.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y,
where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Percent (%) nucleic acid sequence identity” is defined as the percentage of nucleotides in a candidate sequence that are identical with the nucleotides in a reference polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

“Primate” as used herein refers to any of an order of mammals comprising humans, apes, monkeys, and related forms, such as lemurs and tarsiers.

“Purifying” means increasing the degree of purity of a polypeptide in a composition by removing (completely or partially) at least one contaminant from the composition. A “purification step” may be part of an overall purification process resulting in an “essentially pure” composition. An essentially pure composition contains at least about 90% by weight of the polypeptide of interest, based on total weight of the composition, preferably at least about 95% by weight.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “selectively binds” as used herein, refers to the ability of an antibody to adhere to specific antigens and not adhere to other polypeptides. For example, a pan-specific anti-humanized antibody antibody (anti-hzAbAb) selectively binds a group of antibodies having variable domain framework regions that share at least 80% sequence identity and does not bind antibodies with more highly diverse variable domain framework regions.

A “soluble” portion of a polypeptide, as used herein, refers to a portion that is soluble in water and lacks appreciable affinity for lipids (e.g., missing the transmembrane domain or the transmembrane and the cytoplasmic domains).

A “variant” of a polypeptide refers to a polypeptide that contains an amino acid sequence that differs from a reference sequence. The reference sequence can be a full-length native polypeptide sequence or any other fragment of a full-length polypeptide sequence. In some embodiments, the reference sequence is a variable domain heavy chain or variable domain light chain consensus sequence. A polypeptide variant generally has at least about 80% amino acid sequence identity with the reference sequence.

B. Antigen Preparation

A peptide or polypeptide can be directly used as an immunogen to generate antibodies. In some embodiments, at least two polypeptides, for example, at least two antibodies in a mixture can be used as an immunogen. The at least two antibodies can be, for example, humanized antibodies, used as immunogen to generate a pan-specific anti-humanized antibody antibody (anti-hzAbAb). Antibodies useful in an antigen cocktail to generate a pan-specific antibody that selectively binds members of a group of related antibodies can include monoclonal, chimeric, humanized, bispecific, multivalent, and the like antibodies as well as fragments thereof.

To generate a useful pan-specific antibody that selectively binds humanized antibodies containing related variable domain framework regions, the immunogen can comprise two or more humanized antibodies having variable domain framework regions that share at least 80% sequence identify, and may share at least 80% sequence identity with the framework regions of a variable domain consensus sequence. The consensus sequence can be, for example, derived from a compilation of human V_{H} subgroup III or V_{L}κ subgroup I variable domain sequences.

Antibody is raised against antigen derived from a first mammalian species. Preferably the first mammalian species is human. However, other mammals are contemplated such as farm, pet, or zoo animals, e.g. where the antibody is intended to be used to treat such mammals.

To generate an antibody mutant, one or more amino acid alterations (e.g. substitution, deletion, insertion) are made to the amino acid sequence, as known.

An antigen to be used for production of antibodies can be, for example, a soluble form of the full length polypeptide or a fragment thereof, such as a solubilized full length molecule or a fragment. The polypeptide antigen can be an antibody, for example, a full length antibody or an antibody fragment, such as an Fv fragment. An antibody can be raised against an antigen cocktail, a mixture of two or more polypeptides, such as a mixture of at least two related humanized antibodies, having variable domain framework regions that share at least 80% sequence identity. The at least two related humanized antibodies can share at least 80% sequence identity with the framework regions of a variable domain consensus sequence. In one embodiment, an antigen cocktail contains a mixture of at least two related humanized antibodies, each having at least 80% sequence identity with a the framework regions of a variable domain consensus sequence containing at each residue the most abundant amino acid in a compilation of human heavy chain subgroup III or light chain κ subgroup I variable domains. The framework regions can share at least 85%, 90%, or 95% sequence identity, for example.

C. Antibody Production

1. Monoclonal Antibodies. Monoclonal antibodies may be made using any number of methods including the hybridoma method first described by Kohler et al., 1975, Nature, 256:495. Monoclonal antibodies can also be made by recombinant DNA methods (see, for example, U.S. Pat. No. 4,816,567).

Pan-specific monoclonal antibodies that selectively bind humanized antibodies having related variable domain framework regions can be made by immunizing an animal with two or more different humanized antibodies that have related framework regions, that is, sharing at least 80% sequence identity. The related variable domain framework regions can be identical to the variable domain framework regions of a consensus sequence for the heavy and/or light chain variable domain, respectively, or each may have, for example, up to 10 amino acid substitutions, and may have, for example, 1, 2, 3, 4, 5, 6, 7, 8, 9, or 10 amino acid substitutions. In some embodiments, the immunogen can include and preferably includes 3, 4, 5, 6, 7, or 8 different humanized antibodies having related framework regions sharing at least 80% sequence identity. The at least two humanized antibodies generally are directed to different antigens, and generally specifically bind different antigens and/or different antigenic epitopes. For example, a humanized anti-Her2 antibody can be combined with a humanized anti-CD20 antibody in an antigen cocktail, where the humanized antibodies contain variable domain framework regions sharing at least 80% sequence identity and having at least 80% identity with the framework regions of a light chain variable domain kappa subgroup I consensus sequence, and/or with the framework regions of a heavy chain variable domain subgroup III consensus sequence.

In a hybridoma method, a mouse or other appropriate host animal, such as a hamster, is immunized to elicit lymphocytes that produce or are capable of producing antibodies that specifically bind the protein(s) used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, 1984, J. Immunol. 133:3001; and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis described in Munson et al. 1980, Anal Biochem. 107:220.

Pan-specific monoclonal antibodies selected from anti-antibody antibodies produced after immunized with at least two, for example, with 3, 4, 5, 6, 7, or 8 related humanized antibodies or fragments thereof, are selected, for example by immunoassay, as those antibodies that selectively bind at least two, and for example, may bind 3, 4, 5, 6, 7, 8, 9, or 10 antibodies or antibody fragments used in the immunization cocktail or having variable domain framework regions that share at least 80% sequence identity with the antibodies or antibody fragments used in the immunization coctail, and may share at least 85, 90, 95, or 99% sequence identity with the immunizing antibodies or antibody fragments. In some embodiments, a selected pan-specific antibody binds all of the antibodies or antibody fragments present in the immunizing mixture. In other embodiments, the selected pan-specific antibody binds at least two of the antibodies or antibody fragments in the antigen cocktail, and also binds one or more related antibody, for example, having framework regions that share at least 80% sequence identity with the variable domain framework regions of those antibodies or antibody fragments in the antigen cocktail.

Screening methods useful to identify pan-specific antibodies include, for example, ELISA, ECLA, and Biacore analysis. Such screening analysis permits selection of pan-specific antibodies that selectively bind two or more antibodies or antibody fragments that formed the antigen cocktail. Antibodies can be further screened to select preferred pan-specific antibodies with specific binding to humanized antibodies that were not included in the immunizing antigen cocktail, but have variable domain framework regions sharing at least 80% sequence identity. Selection may also include exclusion of those anti-humanized antibodies that bind unrelated antibodies, for example, excluding anti-antibody antibodies that bind human constant domains by screening against a general human antibody pool, for example. Selection may also include screening against chimeric antibodies containing non-human variable domains and human constant domains, for example, to exclude those antibodies that bind to the constant domains.

Once hybridoma cells that produce pan-specific antibodies of a desired specificity, affinity, and/or activity are identified, clones may be subcloned by limiting dilution procedures and grown by standard methods (Goding, Monoclonal Antibodies: Principles and Practice, pp. 59-103 (Academic Press, 1986)). Suitable culture media for this purpose include, for example, DMEM or RPMI-1640 medium. In addition, the hybridoma cells may be grown in vivo as ascites tumors in an animal e.g., by i.p. injection of the cells into mice.

The pan-specific monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the pan-specific monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-262 and Plückthun, 1992, Immunol. Revs. 130:151-188.

In a further embodiment, monoclonal antibodies or antibody fragments can be isolated from antibody phage libraries generated using the techniques described in McCafferty et al., 1990, Nature, 348:552-554. Clackson et al., Nature, 1991, 352:624-628 and Marks et al., 1991, J. Mol. Biol. 222:581-597 describe the isolation of murine and human antibodies, respectively, using phage libraries. Subsequent publications describe the production of high affinity (nM range) human antibodies by chain shuffling), as well as combinatorial infection and in vivo recombination as a strategy for constructing very large phage libraries (Waterhouse et al., 1993, Nuc. Acids. Res. 21 :2265-2266). Thus, these techniques are viable alternatives to traditional monoclonal antibody hybridoma techniques for isolation of pan-specific monoclonal antibodies.

Alternatively, phage display technology (see, for example, McCafferty et al., 1990, Nature 348:552-553) can be used to produce human antibodies and antibody fragments in vitro, from immunoglobulin variable domain gene repertoires from non-immunized donors. According to this technique, genes encoding antibody variable domains are cloned in-frame into either a major or minor coat protein gene of a filamentous bacteriophage, such as M13 or fd, and displayed as functional antibody fragments on the surface of the phage particle. Since the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson et al., 1993, Current Opinion in Structural Biology 3:564-571.

Several sources of variable domain gene segments can be used for phage display. Clackson et al., 1991, Nature 352:624-628 isolated a diverse array of anti-oxazolone antibodies from a small random combinatorial library of variable domain genes derived from the spleens of immunized mice. A repertoire of genes encoding variable domains from non-immunized human donors can be constructed and antibodies to a diverse array of antigens (including self-antigens) can be isolated essentially following the techniques described by Marks et al., 1991, J. Mol. Biol. 222:581-597, or Griffith et al., 1993, EMBO J. 12:725-734. See, also, U.S. Pat. Nos. 5,565,332 and 5,573,905.

A phage display library of antibody variable domains or antigen binding fragments can be screened for binding to at least two humanized antibodies. For example, a phage display library can be screened for antibody variable domains that bind to an anti-Her2 humanized antibody and an anti-VEGF humanized antibody. In some embodiments, additional humanized antibodies can serve as target antigens, preferably up to 10 antibodies, more preferably 3 to 6 antibodies. In some embodiments, it is desirable that a pan-specific antibody or antigen-binding fragment binds at least two and may bind, for example, 3, 4, 5, 6, 7, or 8 different humanized antibodies having related variable domain framework regions.

Alternatively, genes encoding variable domain framework region such as the framework regions of a variable domain consensus sequence, or fragments thereof can be fused in frame to a phage major or minor coat protein. In one embodiment, variable light chain kappa subgroup I or a variable heavy chain subgroup III framework regions or fragment thereof can be displayed by phage as described above. A library of framework region polypeptides and variant polypeptides, for example related framework regions having at least 80% sequence identity, including variant framework regions having, for example 1 to 10 amino acid substitutions, can be screened by well known methods by candidate pan-specific anti-hzAbAb. Screening a phage display library displaying variable domain framework region polypeptides can be a useful method to screen antibodies against multiple variable domain framework region sequences, such as those antibodies that selectively bind related variable domain framework regions having at least 80% identity to a human consensus variable domain framework region.

DNA encoding an antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567; and Morrison, et al., 1984, Proc. Nat'l Acad. Sci. USA 81:6851). Alternatively, the immunoglobulin coding sequence can be fused with all or part of a sequence encoding a non-immunoglobulin polypeptide (heterologous polypeptide). The non-immunoglobulin polypeptide sequences can substitute for the constant domains of an antibody, or can substitute for the variable domains of one antigen-combining site of an antibody to create a chimeric bivalent antibody comprising one antigen-combining site having specificity for an antigen and another antigen-combining site having specificity for a different antigen.

2. Humanized Antibodies. Methods for humanizing nonhuman antibodies have been described in the art. Preferably, a humanized antibody has one or more nonhuman amino acid residues introduced into its sequence. These nonhuman amino acid residues are often referred to as “import” or “donor” residues that are typically taken from an “import” or “donor” variable domain and introduced into a human “recipient” sequence. Humanization can be performed by substituting hypervariable region sequences (CDRs or HVLs) from nonhuman sources for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a nonhuman species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity and human anti-mouse antibody (HAMA) response when the antibody is intended for human therapeutic use. According to the so-called “best-fit” method, the sequence of the variable domain of a rodent antibody is screened against the entire library of known human variable domain sequences. The human variable domain sequence, which is closest to that of the rodent, is identified and the human variable domain framework region (FR) within it is accepted for the humanized antibody (Sims et al., 1993, J. Immunol. 151:2296; Chothia et al., 1987, J. Mol. Biol., 196:901). Another method uses a particular framework region derived from a consensus sequence variable domain. The consensus sequence can be delineated by residues identified in a compilation of variable domain residues for known human antibodies in a particular subgroup of light or heavy chains. The same variable domain framework region may be used to produce several different humanized antibodies (Carter et al., 1992, Proc. Natl. Acad. Sci. USA 89:4285; Presta et al., 1993, J. Immunol. 151 :2623).

Antibodies can be humanized with retention of high binding affinity for the antigen and other favorable biological properties. To achieve this goal, according to a preferred method, humanized antibodies are prepared by a process that includes analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, variable domain framework region residues can be selected and combined from the recipient and import sequences so that the desired antibody characteristic, such as increased affinity for the target antigen(s), is achieved. In general, the hypervariable region residues are directly and most substantially involved in influencing antigen binding.

Although the variable domain framework regions of multiple humanized therapeutic antibodies can be identical to the sequences of a consensus sequence, the framework regions (FRs) of each humanized antibody sequence may differ from the FRs of the consensus sequence. Amino acid substitutions in the framework regions can be made, for example, to increase affinity to an antigen. See, for example, WO 98/45331. Substituting mouse residues or alternative human residues can increase affinity of the antibody to the antigen. Substitutions can be made to either the heavy or light chain variable domain. Most FR substitutions occur in the heavy chain variable domain framework region 3 (FR3). Thus, the consensus framework regions provide a basis for constructing a humanized antibody.

The humanized antibody may be an antibody fragment, such as a Fab, optionally conjugated with one or more cytotoxic agent(s) in order to generate an immunoconjugate. Alternatively, the humanized antibody may be an full length antibody, such as an full length IgGI antibody.

3. Antibody Fragments. In certain circumstances it may be advantageous to use antibody fragments, rather than whole antibodies in therapeutic treatments. The smaller size of the fragments allows for rapid clearance, and may lead to improved access to solid tumors, for example. These antibody fragments can be therapeutic products having related variable domain framework regions to be recognized by a pan-specific anti-hzAbAb.

Various techniques have been developed for the production of antibody fragments. Traditionally, fragments are derived via proteolytic digestion of intact antibodies (see, e.g., Morimoto et al., 1992, Journal of Biochemical and Biophysical Methods 24:107-117; and Brennan et al., 1985, Science, 229:81). However, fragments can now be produced directly by recombinant host cells. Fab, Fv and ScFv antibody fragments can all be expressed in and secreted from E. coli, allowing the facile production of large amounts of these fragments. Alternatively, Fab′-SH fragments can be directly recovered from E. coli and chemically coupled to form F(ab′)₂ fragments (Carter et al., 1992, Bio/Technology 10: 163-167). In another approach, F(ab′)₂ fragments can be isolated directly from recombinant host cell culture. Fab and F(ab′)₂ fragments with increased in vivo half-life comprising a salvage receptor binding epitope residues are described in U.S. Pat. No. 5,869,046. Other techniques for the production of antibody fragments will be apparent to the skilled practitioner. An antibody of choice can be a single chain Fv fragment (scFv). See WO 93/16185; U.S. Pat. No. 5,571,894; and U.S. Pat. No. 5,587,458. Fv and sFv are the only species of fragments with intact combining sites that are devoid of constant regions; thus, and are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

Humanized antibody fragments, such as Fab fragments, can also be used in mixtures of humanized antibodies to immunize an animal and generate pan-specific anti-hzAbAb. Antibody fragments contain at least heavy and light chain variable domains. An antibody fragment, such as an scFv, has V_{H} and V_{L} can comprise variable domains framework regions showing at least 80% sequence identity with the FRs of a variable domain consensus sequence. Thus, a pan-specific anti-hzAbAb binds humanized antibody fragment just as a pan-specific anti-hzAbAb would bind a full-length antibody.

4. Bispecific Antibodies. Bispecific antibodies are antibodies that have binding specificities for at least two different epitopes. Exemplary bispecific antibodies may bind to two different epitopes of a protein. Other such antibodies may combine a one antibody binding site with a binding site for another protein. Alternatively, one antibody arm may be combined with an arm that binds to a triggering molecule on a cell. Bispecific antibodies may also be used to localize cytotoxic agents to cells. Such antibodies possess one therapeutic arm and another arm that binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in W098/02463. U.S. Pat. No. 5,821,337 teaches a bispecific anti-ErbB2/anti-CD3 antibody.

Antibodies with more than two valencies are contemplated. For example, trispecific antibodies can be prepared. See Tutt et al., 1991, J. Immunol. 147:60.

5. Multivalent Antibodies. A multivalent antibody may be internalized (and/or catabolized) faster than a bivalent antibody by a cell expressing an antigen to which the antibodies bind. Antibodies useful in an antigen cocktail for producing pan-specific antibodies can be multivalent antibodies (other than of the IBM class) with three or more antigen binding sites (e.g. tetravalent antibodies) that can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: V_{H}-C_{H} 1-flexible linker-V_{H}-C_{H}1-Fc region chain; or V_{H}-C_{H}1-V_{H}-C_{H}1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a C_{L} domain.

6. Polyclonal antibodies. Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, and the like.

Animals can be immunized against an antigen, an antigen cocktail, immunogenic conjugates, or derivatives by combining, for example, 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

D. Selection and Transformation of Host Cells

Suitable host cells for cloning or expressing the recombinant polypeptides, including monoclonal antibodies described herein are prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Full length antibody, antibody fragments, and antibody fusion proteins useful in the antigen cocktail can be produced in bacteria. Production in E. coli is fast and cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g., in CHO cells.

In addition to prokaryotes, eukaryotic microbes such as filamentous fungi or yeast are suitable cloning or expression hosts for antibody-encoding. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for expression are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., 1977, J. Gen Virol. 36:59); baby hamster kidney cells (BHK, ATCC CCL 10); Chinese hamster ovary cells/-DHFR (CHO, Urlaub et al., 1980, Proc. Natl. Acad. Sci. USA 77:4216); mouse sertoli cells (TM4, Mather, 1980, Biol. Reprod. 23:243-251; monkey kidney cells (CV1 ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (WI38, ATCC CCL 75); human liver cells (Hep G2, HB 8065); mouse mammary tumor (MMT 060562, ATCC CCL5 1); TRI cells (Mather et al., 1982, Annals N.Y. Acad. Sci. 383:44-68); MRC 5 cells; FS4 cells; and a human hepatoma line (Hep G2).

Host cells are transformed with expression or cloning vectors for a B cell depleting antibody such as CD20 binding antibody, or an integrin antagonist antibody production and cultured in conventional nutrient media modified as appropriate for inducing promoters, selecting transformants, or amplifying the genes encoding the desired sequences.

The host cells used to produce a pan-specific antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., 1979, Meth. Enz. 58:44, Barnes et al., 1980, Anal. Biochem. 102:255, U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (e.g., gentamicin), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

E. Purification of Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., 1992, Bio/Technology 10: 163-167 describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., 1983, J. Immunol. Meth. 62:1-13). Protein G is recommended for all mouse isotypes and for human γ3 (Guss et al., 1986, EMBO J. 5:15671575). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin sepharose chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

F. Screening Assays

Methods for detecting a pan-specific anti-hzAbAb include analyzing an antibody sample for selective binding to at least two related antigens. For example, methods for detecting a pan-specific antibody include analyzing for antibody binding to two or more humanized antibodies containing variable domain framework regions that share at least 80% sequence identity. A pan-specific antibody is identified as an antibody that selectively binds the at least two humanized antibodies having related variable domain framework regions. The methods for identifying the pan-specific antibody can utilize binding assays known to those of skill in the art including ELISA, ECLA, RIA, BIAcore™, and the like.

1. ELISA. Immunoassay systems include, for example, solid-phase ELISA and capture ELISA. In a capture ELISA, immobilization of a polypeptide to a solid phase is accomplished by known methods. A polypeptide may be absorbed onto a solid phase that comprises an assay surface or matrix (see, for example, U.S. Pat. No. 3,720,760). A polypeptide antigen such as an antibody can be coupled, non-covalently or covalently to an assay surface, with or without prior activation of the support. Deposit of a polypeptide as a capture reagent can also be by immunoprecipitation, for example, after binding the sample antibody. In one embodiment, a humanized antibody is immobilized by diluting the polypeptide solution to below the critical micelle value. Deposition of the soluble protein onto an assay surface can be achieved in slightly denaturing conditions, for example, mildly basic or acidic conditions. Alternatively, the protein can be captured by a covalent linkage at the assay surface, or bound by a protein such as an antibody disposed on the assay surface. The capture reagent can be a humanized antibody.

The solid phase used for immobilization may be any inert support or carrier that is essentially water insoluble and useful in immunoassays, including supports in the form of, for example, surfaces, particles, porous matrices, and the like. Examples of commonly used supports include small sheets, Sephadex®, polyvinyl chloride, plastic beads, microparticles, assay plates, test tubes manufactured from polyethylene, polypropylene, polystyrene, and the like. Such supports include 96-well microtiter plates, as well as particulate materials such as filter paper, agarose, cross-linked dextran, and other polysaccharides. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are suitably employed for capture reagent immobilization. The immobilized capture reagent can be coated on a microtiter plate. The solid phase can be a multiwell microtiter plate that can be used to analyze several samples at one time.

The solid phase is coated with the capture reagent that may be linked by a non-covalent or covalent interaction or physical linkage, as desired. Techniques for attachment include those described in U.S. Pat. No. 4,376,110 and the references cited therein.

If polystyrene or polypropylene plates are utilized, the wells in the plate can be coated with the capture reagent (typically diluted in a buffer such as 0.05 M sodium carbonate) by incubation for at least about 10 hours, for example, overnight, at temperatures of about 4-20° C., for example 4-8° C., and at a pH of about 8-12, for example in the range of 9-10 or about 9.6. If shorter coating times (1-2 hours) are desired, the plate can be coated at 37° C. or contain nitrocellulose filter bottoms, for example, Millipore Multiscreen™ (Billerica, Mass.) can be used. A capture reagent may be applied to an assay surface as soluble proteins in detergent. Dilution of the detergent to below the critical micelle value will cause the polypeptide to precipitate on the assay surface.

Coated plates are typically treated with a blocking agent that binds non-specifically to and saturates the binding sites to prevent unwanted binding of free ligand to excess binding sites on the wells of the plate. A blocking treatment typically takes place under conditions of ambient temperatures for about 1-4 hours, for example, in the range of 1.5 to 3 hours.

After coating and blocking, a serum sample to be analyzed is diluted as necessary and added to the immobilized phase. The dilution rate is generally about 5-15%, for example 10%, by volume. For sufficient sensitivity, the immobilized capture reagent can be in molar excess of the maximum molar concentration of the analyte anticipated in the sample after appropriate dilution. Conditions for incubation of sample and capture reagent are selected to maximize sensitivity of the assay and to minimize dissociation. Incubation time depends primarily on the temperature. The sample is separated from the immobilized capture reagent with a wash solution to remove uncaptured analyte from the system. The wash solution is generally a buffer.

In general, the system can be washed three times. The temperature of the wash solution is typically from about 0-40° C., for example, in the rage of 4-30° C. An automated plate washer may be utilized. A cross-linking agent or other suitable agent may be added to the wash solution to covalently attach the captured analyte to the capture reagent.

Following removal of uncaptured analyte molecules from the system, captured analyte molecules are contacted with a detection reagent, such as an antibody, for example, at room temperature.

The temperature and time for contacting the analyte with the detecting agent is dependent primarily on the detection means employed. For example, when horseradish peroxidase (HRP) conjugated to sheep anti-mouse IgG is used as the means for detection, the detecting agent can be incubated with the captured analyte for about 0.5-2 hours, for example, about 1 hour. The system is washed as described above to remove unbound detecting agent from the system and developed by adding peroxidase substrate and incubating the plate for about 5 minutes at room temperature or until good color is visible.

A molar excess of the detection reagent can be added to the system after the unbound analyte has been washed from the system. A detection reagent may be a polyclonal or monoclonal antibody, or a mixture thereof. The detection reagent may be directly or indirectly detectable.

The affinity of the detection reagent is sufficiently high such that amounts of analyte can be detected. A fluorimetric or chemiluminescent label moiety has greater sensitivity in immunoassays compared to a conventional calorimetric label. The binding affinity of the selected detection reagent must be considered in view of the binding affinity of the capture agent such that the detection reagent does not strip the analyte from the capture reagent.

The label moiety is any detectable functionality that does not interfere with the binding of the captured analyte to the detecting agent. Examples of suitable label moieties include moieties that may be detected directly, such as fluorochrome, chemiluminscent, and radioactive labels, as well as moieties, such as enzymes, that must be reacted or derivatized to be detected. Examples of such labels include, but are not limited to radioisotopes ³²P, ¹⁴C, ¹²⁵I, ³H, and ¹³¹I, fluorophores such as rare earth chelates or fluorescein and its derivatives, rhodaimine and its derivatives, luceriferases, e g., firefly luciferase and bacterial luciferase (U.S. Pat. No. 4,737,456), horseradish peroxidase (HRP), alkaline phosphatase, an enzyme that employs hydrogen peroxide to oxidize a dye precursor such as HPP, biotin/avidin, biotin/streptavidin, and the like.

Conjugation of the label moiety to the detecting agent, such as for example an antibody, is a standard manipulative procedure in immunoassay techniques. See, for example, O'Sullivan et al. “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166. Conventional methods are available to bind the label moiety covalently to proteins or polypeptides.

The amount of analyte bound to the capture reagent can be determined by washing away unbound detecting agent from the immobilized phase and measuring the amount of detecting agent bound to the analyte using a detection method appropriate to the label. In an embodiment, the label moiety is an enzyme. In the case of enzyme moieties, the amount of developed color is a direct measurement of the amount of captured analyte. For example, when HRP is the label moiety, color is detected by quantifying the optical density (O.D.) at 650 nm absorbance. In another embodiment, the quantity of analyte bound to the capture reagent can be determined indirectly.

2. BIAcore™ Binding Analysis. Binding affinities of full length antibodies can be calculated from association and disassociation rate constants measured using a BIAcore™-3000 surface plasmon resonance system (BlAcore, Inc., Piscataway, N.J.) as described in Chen et al., 1999, J. Mol. Biol. 293:865-881. A biosensor chip is activated for covalent coupling of a target polypeptide using N-ethyl-N′-(3-dimethylaminopropyl)-carbo-dimide hydrochloride (EDC) and N-hydroxysuccinimid (NHS) according to the supplier's (BlAcore, Inc.) instructions. A target polypeptide can be buffer-exchanged into 20 mM sodium acetate (pH 4.8) and to achieve approximately 700-1400 response units (RU) of coupled protein. A solution of 1 M ethanolamine can be injected as a blocking agent.

For kinetics measurements, serial dilutions of full length antibodies can be injected in PBS/Tween® buffer (0.05% Tween®-20 in phosphate-buffered saline) at 25° C. or 37° C. at a flow rate of 10 μl/minute. Equilibrium dissociation constants, K_{D} values from SPR measurements can be calculated as k_{off}/kₒₙ.

Methods of determining binding affinities by BIAcore's surface plasmon resonance system are further disclosed in U.S. Pat. Nos. 5,641,640; 5,955,729; 5,965,456; and 6,143,574, which are hereby incorporated by reference.

3. ECLA. Conventional methods for ECLA can be used to screen hybridoma clones for potential pan-specific anti-hzAbAb. See, for example, U.S. Pat. Nos. 5,543,112; 5,935,779; 6,316,607, the patents referenced therein. The capture reagent and detecting reagent can be mixed with the analyte molecule and incubated at room temperature. In an embodiment, the capture reagent and detecting reagent are in molar excess of the maximum molar concentration of the analyte molecule anticipated in the sample. Depending on the analyte molecule, the capture reagent may compete for binding sites with the detecting reagent yielding inaccurate results. Therefore, the final concentration of the capture reagent will normally be determined empirically to maximize the sensitivity of the assay over the range of interest. In an embodiment, the capture reagent and detecting reagent are added to the sample in about a 1:1 ratio.

The capture reagent can be an antigen, receptor, antibody, or fragment thereof. Preferably the antibody is monoclonal. In an embodiment, the capture reagent is an antibody such as a humanized therapeutic antibody. The detecting agent can be a receptor, antibody, or fragment thereof, and can be a monoclonal antibody. The monoclonal antibody can be a murine monoclonal antibody.

Incubation of the capture reagent, detection agent, and analyte can occur for about 0.5 to 3 hours, preferably 1.5-3 hours at 36-38° C. The pH of the incubation buffer is chosen to maintain a significant level of specific binding of the capture reagent and detecting agent to the analyte. In an embodiment, the pH of the incubation buffer is about 6-9.5, more preferably about 6-7. Various buffers can be employed to achieve and maintain the desired pH during this step, including borate, phosphate, carbonate, Tris-HCl or Tns-phosphate, acetate, barbital, and the like. The particular buffer employed is usually not critical, however, in individual assays one buffer may be preferred over another.

In an embodiment, the ECLA method utilizes a binding phase to immobilize the analyte complex, such as beads or microparticles. The beads or microparticles can have a diameter of 0.05 μm to 200 μm, more preferably 0.1 um to 100 um, more preferably 0.5 um to 10 um, and a surface component capable of binding the capture reagent. In an embodiment, the binding surface of the beads or microparticles is coated with streptavidin and the capture reagent is labeled with biotin. The microparticles can also be coated, for example, with glutathione, anti-IgG antibody, or agglutinin. The capture reagent can be biotinylated with biotinylamidocaproic acid-N-hydroxysuccinimide ester using standard amine chemistry at a ratio from about 1:1 to about 10:1 biotin to capture reagent, more preferably from about 2:1 to about 4:1 biotin to capture reagent, more preferably about 2.5:1 biotin to capture reagent.

After incubating the capture reagent and detecting reagent with the analyte molecule, microparticles capable of binding the capture reagent are added to the mixture and the mixture is incubated. In an embodiment, the microparticles are coated with a molecule that binds biotin, such as streptavidin. The incubation time can be from about 0.5 to 3 hours, preferably 1.5-3 hours at 36-38° C. The pH of the incubation buffer is chosen to maintain a significant level of specific binding of the capture reagent/detecting agent/analyte molecule complex to the microparticles. The pH of the incubation buffer can be about 6-9.5, more preferably about 6-7. The incubation buffer can include an electrolyte. The electrolyte can be one or more salts or other species. In an embodiment, the electrolyte is a sodium salt or potassium salt.

The microparticles are assayed with an apparatus that contains an electrode and a photodetector, such as an IGEN M384 analyzer (IGEN International Inc., Gaithersburg, Mass.). See, for example, U.S. Pat. Nos. 5,543,112 and 5,935,779 describing apparatuses for measuring electrochemiluminescence. The label conjugated to the detecting agent is induced to emit electromagnetic radiation by stimulating the label into an excited state. Detection and/or quantitation of the analyte in a sample is typically made by comparing the luminescence of a sample to the luminescence emitted by a calibration standard developed with known amounts of the analyte and detecting agent. In an embodiment, the photodetector measures the light emitted by the label and software for analyzing data collected by the photodetector is used to calculate the concentration of analyte molecular or ECLA response (in electrochemiluminescence units (ECLU)) of the analyte molecule.

In an embodiment, the label conjugated to the detecting reagent is a metal chelate that luminesces under the electrochemical conditions imposed by ECLA. The metal can be, for example, a transition metal (such as a d-block transition metal) or a rare earth metal. In an embodiment, the metal is ruthenium, osmium, rhenium, iridium, rhodium, platinum, indium, palladium, molybdenum, technetium, copper, chromium, or tungsten. In an embodiment, the metal is ruthenium or osmium.

A ligand(s) linked to the metal of the chelate is usually heterocyclic or organic in nature, and plays a role in determining whether the metal chelate is soluble in an aqueous environment or in an organic or other nonaqueous environment. The ligands can be polydentate, and can be substituted. Polydentate ligands include aromatic and aliphatic ligands. Suitable aromatic polydentate ligands include aromatic heterocyclic ligands. In an embodiment, the aromatic heterocyclic ligands are nitrogen-containing, such as, for example, bipyridyl, bipyrazyl, terpyridyl, and phenanthrolyl. Suitable substituents include for example, alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, sulfur-containing groups, phosphorus containing groups, and the carboxylate ester of N-hydroxysuccinimide. The chelate can have one or more monodentate ligands, a wide variety of which are known to the art. Suitable monodentate ligands include, for example, carbon monoxide, cyanides, isocyanides, halides, and aliphatic, aromatic and heterocyclic phosphines, amines, stilbenes, and arsines.

Examples of suitable chelates are bis[(4,4′-carbomethoxy)-2,2′-bipyridine]2-[3-(4-methyl-2,2′-bipyridine-4-yl)propyl]-1,3-dioxolane ruthenium(II); bis(2,2′bipyridine)[4-(butan-1-al)-4′-methyl-2,2′-bipyridine]ruthenium(II); bis(2,2′-bipyridine)[4-(4′methyl-2,2′-bipyridine-4′-yl)-butyric acid]ruthenium(II); tris(2,2′bipyridine)ruthenium(II); (2,2′-bipyridine)[bis-bis(1,2-diphenylphosphino)ethylene]2-[3-(4-methyl-2,2′-bipyridine-4′-yl)propyl]-1,3-dioxolane osmium(II); bis(2,2′-bipyridine)[4-(4′-methyl-2,2′-bipyridine)-butylamine]ruthenium(II); bis(2,2′-bipyridine)[1-bromo-4(4′-methyl-2,2′-bipyridine-4-yl)butane]ruthenium(II); bis(2,2′-bipyridine)maleimidohexanoic acid, 4-methyl-2,2′-bipyridine-4′-butylamide ruthenium(II). Additional label moieties suitable for ECLA are described in U.S. Pat. Nos. 5,591,581; 6,271,041; 6,316,607; and 6,451,225. In an embodiment, the label moiety is Ru(bpy)₃²⁺ or ORI-TAG™ NHS ester (IGEN International Inc., Gaithersburg, Mass.).

The amount of label utilized is that amount which effectively results in the emission of a detectable, and if desired, quantifiable, emission of electromagnetic energy. In an embodiment, the detecting agent is conjugated with the label, using standard amine chemistry, at a ratio from about 1:1 to about 10:1 label to detecting reagent, more preferably from about 3:1 to about 7:1 label to detecting reagent, more preferably about 5:1 label to detecting reagent.

The pH and/or temperature of the system can be varied to identify molecules that bind the target molecule.

G. Methods Using a Pan-Specific Anti hzAbAb

With an ever growing amount of antibodies being used as therapeutics, for example, Avastin™ by Genentech Inc., Zenapax® by Protein Design Labs, Zevalin® by Biogen IDEC, Inc., Campath® by Genzyme Oncology, etc., there is a need for reagents to recognize these biologic therapeutics in pharmacokinetic and pharmacodynamic assays and for quality control. Humanized antibodies can be efficiently produced using identical variable domain framework regions substituted with different hypervariable regions (CDRs or HVLs). For instance, Genentech, Inc. uses a consensus variable domain framework region sequence as the basis for its humanized antibodies (see, for example, Carter, U.S. Pat. No. 6,054,297.

As described in the Examples below, pan-specific antibodies have now been developed that recognize a panel of Genentech's humanized antibodies, variable domain framework regions that share at least 80% sequence identity but contain differing hypervariable regions (CDRs and HVLs). These pan-specific antibodies do not recognize a chimeric or a generic human antibody. An antibody specific to a company's therapeutic antibodies is a useful reagent for preclinical and clinical testing.

Pan-specific anti-humanized antibody antibodies (anti-hzAbAb) generated by methods described herein can be used to develop a generic pharmacokinetic (PK) assay that measures drug concentration, for example, a therapeutic humanized antibody, in preclinical studies of a humanized therapeutic antibody replacing single antigen-specific reagents. Use of a pan-specific reagent reduces and streamlines PK assay development time and costs for developing individual PK assays for each molecule of interest. In the absence of target-specific reagents that are generally used for each individual PK assays, a pan-specific reagent can provide a generic assay.

Pan-specific anti-hzAbAbs generated as described herein can also be used to develop a total or bound therapeutic drug PK assay, for example, where the drug is a therapeutic humanized antibody.

Target specific reagents such as anti-CDR antibodies raised against therapeutic humanized antibody drugs or the ligand targets of the antibody drug are useful to develop a free drug PK assay. However, these assays are limited in the total or bound drug PK assay development, wherein the drug is a therapeutic humanized antibody. Total humanized antibody concentration could be used to quantify total humanized antibody exposure in toxicity studies, and determine the PK and ADME (adsorption, distribution, metabolism, and excretion) of the molecule. A pan-specific anti-hzAbAb would be very useful in such assays. A pan-specific antibody useful for this purpose will bind a region of the molecule shared by the molecules, for example, the shared framework regions.

Additionally, when mutant humanized antibodies are generated for further selection of a drug candidate, preclinical studies are useful to assist the process. PK profiles of the mutants from these preclinical studies usually need to be considered. Since mutations are usually made in CDR regions, target-specific reagents (such as anti-CDR antibodies or ligand of the drug) lack consistent responses to various mutants. Assay conditions then need to be optimized for each mutant PK assay. A pan-specific anti-hzAbAb that binds to the variable domain framework region provides a reagent that is suitable for all mutants containing a related variable domain framework region.

Pan-specific hzAbAbs generated in this process can also be used to measure monoclonal antibody concentration. Changing hypervariable regions (CDRs or HVLs) may change extinction coefficients, and therefore, using just UV spectorphotometry may only provide approximate concentrations for various mutants.

Pan-specific anti-hzAbAbs generated in this process can also be used to characterize anti-drug antibody assays. Anti-drug antibody assays are routinely used to assess immunogenicity of a therapeutic humanized antibody drug. Positive controls such as monoclonal anti-drug antibodies generated from a hybridoma process along with polyclonal anti-drug antibodies are used to characterize assay sensitivity, drug interference, etc. Since various positive controls used in assay characterizations may give different numeric results, pan-specific anti-hzAbAbs can therefore provide a standard positive control to assess assay performance across various projects.

A pan-specific anti-hzAbAb is also useful in clinical studies. A pan-specific anti-hzAbAb provides a reagent for toxicity, PK, and ADME studies. Additionally, a pan-specific anti-hzAbAb provides a reagent for assays to monitor a humanized antibody drug in a particular patient. Assays to monitor a humanized antibody drug in a particular patient could be performed in private laboratories and/or physician offices.

The invention may be better understood with reference to the following examples that are exemplary and do not serve to limit the invention in any way.

### EXAMPLES

The invention is further described by reference to the following examples that are exemplary in nature and are not intended to limit the scope of the invention in any way.

### Example 1

### Generation of Anti-humanized Antibody Antibodies (Anti-hzAbAb)

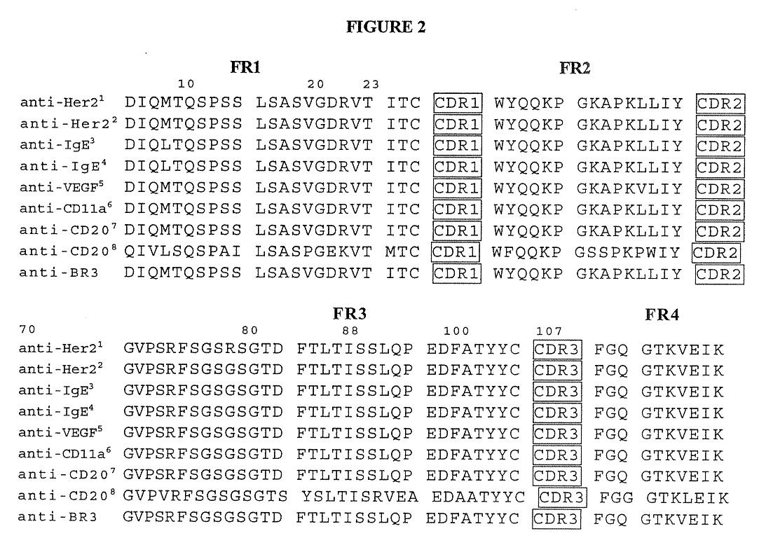

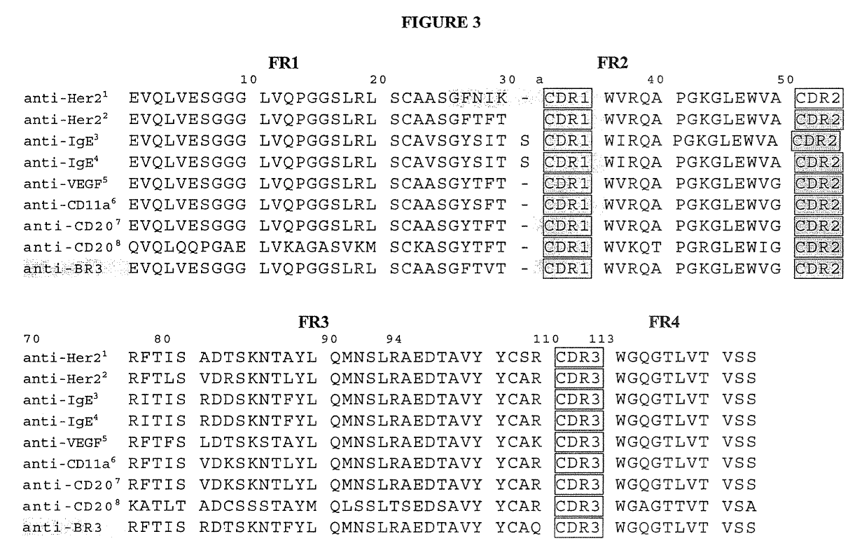

The humanized antibodies described for this Example contain variable domain framework (FR) regions derived from consensus variable domain sequences, with amino acid substitutions made in the consensus sequence framework region residues as suggested by modeling and structure-function analysis. The variable domain framework regions of these humanized antibodies, shown in FIGS. 2 and 3, share at least 80% amino acid sequence identity. The variable domain framework regions of these antibodies also have at least 80% sequence identity with the framework regions of variable domain consensus sequences derived from Kabat compilations of variable domain sequences. To generate pan-specific antibodies that selectively bind at least two of these related humanized antibodies, an antigen cocktail containing a mixture of these antibodies was used to generate anti-humanized antibody antibodies (anti-hzAbAb).

The antigen cocktail contained a mixture of 2 μg each of the following humanized monoclonal IgGI antibodies: anti-VEGF (HuA4.6.1, described, for example in PCT Patent Application WO 98/45331), anti-Her2 (Hu4D5-8, described, for example, in U.S. Pat. No. 5,821,337 and Hu2C4, described, for example in U.S. Pat. No. 6,949,245), anti-CD11a (HuMHM24, described, for example, in U.S. Pat. No. 6,037,454), anti-IgE (E25 and E27, described, for example, in U.S. Pat. No. 6,1725,213), and anti-CD20 antibodies (2H7, described, for example, in U.S. Pat. Appl. Pub. No. 2005/0163775). Each of the antibodies used in the antigen cocktail contains a light chain variable domain having framework regions derived from a variable domain consensus sequence having at each residue the most abundant amino acid identified in a Vκ Subgroup I (VκI) sequence compilation of Kabat et al. (1987, Sequence of Proteins of Immunological Interest, 4^{th} Ed., Public Health Services; 1991, Sequence of Proteins of Immunological Interest, 5^{th} Ed., Public Health Services). Each of the humanized antibodies in the antigen cocktail also contains a heavy chain variable domain consensus sequence having framework regions derived from a variable domain consensus sequence having at each residue the most abundant amino acid identified in a Subgroup III (V_{H}III) sequence compilation of Kabat et al, Supra. The variable domain framework regions of the humanized antibodies in the antigen cocktail mixture are not identical, as one or more amino acid substitutions in the framework regions have been made (see FIGS. 2 and 3).

The suspended mixture of related humanized antibodies was resuspended in monophosphoryl-lipidA and trehalose dicorynomycolate (MPL™+TDM) adjuvant (Corixa, Hamilton,MT) and injected in each hind footpad of a BALB/c mouse at 3 to 4 days intervals. Initial sera were titered against the mixture of antibodies to ensure a good immune response was achieved. Three days after final boost, popliteal lymph nodes were fused with myeloma cell line P3X63Ag.U.1 (see, for example, Chuntharapai et al., 1997, Methods Enzymol. 288: 15-27). Fused cells were selected from unfused myeloma cells by hypoxanthin-aminopterin-thymidine (HAT) selection using a ClonaCell® hybridoma selection kit (StemCell Technologies, Inc., Vancouver, BC).

Clones were isolated and grown to screen for potential pan-specific anti-hzAbAb. The clones were generated against an antigen cocktail containing a mixture of related humanized antibodies to maximize the chances of isolating a pan-specific anti-hzAbAb. It was hypothesized that an antibody selectively binding framework regions derived from a human variable domain consensus sequence might be obtained. As described more fully below in Example 2, screening of 864 isolated hybridoma clones yielded two clones producing anti-hzAbAb demonstrating selective binding to the humanized antibodies of the antigen cocktail as well as to other humanized antibodies having related variable domain framework regions derived from a variable domain consensus sequence formed of the most abundant residues in a Kabat compilation of variable domain residues.

### Example 2

### Screening for Pan-specific Anti-humanized Antibody Antibodies (anti-hzAbAb)

Hybridoma clones generated as described in Example 1 were screened for production of monoclonal antibodies with binding responses to target antibodies having related variable domain framework regions. The variable domains of the different humanized antibodies of the antigen cocktail described for Example 1 generally contain distinct hypervariable regions (CDRs or HVLs) and related variable domain framework regions derived from a variable domain consensus sequence containing the most abundant residues listed in a Kabat compilation. A chimeric anti-CD20 antibody (2B8, described, for example, in U.S. Pat. No. 5,736,137) containing human constant regions but murine variable domains, was used as a negative control during the hybridoma screening, In general, supernatants from hybridoma clones that bound the related humanized antibodies of the antigen cocktail and failed to bind the chimeric anti-CD20 antibody control were considered to be potential candidate pan-specific anti-hzAbAb. Antibody screening was analyzed by ELISA and by ECLA.

ELISA Screening

To screen the 864 clones generated in Example 1, ELISA was performed generally as described in Baker et al., 2002, Trends Biotechnol., 20:149-156. Assays were performed in both 394 and 96 well plates. Briefly, a 384 (or a 96) well plate was coated with 50 μl of goat anti-human IgG Fc (MP Biomedicals, Irvine, Calif.) at a concentration of 2 μg/ml in coating buffer (0.5 M carbonate buffer, pH 9.6). The plates were sealed and stored at 4° C. overnight. After removing the coating solution, 70 μl (or 200 μl for a 96 well plate) of blocking solution containing 0.5% bovine serum albumin and 0.05% Tween®-20 in PBS (pH 7.4) was added to each well. The plates were incubated at room temperature for one hour with agitation and then washed three times with PBS/0.05% Tween®-20.

After the washing step, a 50 μl (or 100 μl for a 96 well plate) of antigen solution (0.4 μg/ml of monoclonal antibody in PBS containing 0.5% of bovine serum albumin and 0.05% Tween®-20) was added to each well, and the plate was incubated at room temperature for one hour with agitation. The plate was washed three times with PBS/0.05% Tween®-20. Then 30-50 μl (or 100 μl for a 96 well plate) of supernatant from individual hybridoma clones was added such that each well in the plate contained supernatant from a single hybridoma clone. The plate was incubated for one hour at room temperature with agitation and washed three times with PBS/0.05% Tween®-20.

After the washing step, 50 μl (or 100 μl for a 96 well plate) of a 1:1000 dilution of sheep anti-mouse IgG coupled to horseradish peroxidase (no cross reactivity to human IgG (MP Biomedicals)) in PBS containing 0.5% bovine serum albumin/0.05% Tween®-20 was added to each well. The plate was incubated at room temperature for one hour with agitation, washed three times with PBS/0.05% Tween®-20, and patted dry. The plate was developed by adding 50 μl (or 100 μl for a 96 well plate) of tetramethylbenzidine (TMB) Microwell Peroxidase substrate (BioFX Laboratories, Owing Mills, Md.) to each well in the plate and incubating for 5-10 minutes at room temperature or until a good color was visible. Development was stopped by adding 50 μl (or 100 μl for a 96 well plate) of Stop solution (BioFX Laboratories, Catalog No. TMBT-0100-01) to each well. Plates were read on a Sunrise plate reader (Tecan US, Inc., Research Triangle Park, N.C.) at 650 nm.

Binding of the candidate anti-hzAbAb to target humanized antibodies was analyzed using antibodies that were part of the antigen cocktail described in Example 1. As an initial screen, all clones were analyzed for binding to humanized anti-VEGF (WO 98/45331), and to two different humanized anti-Her2 antibodies (U.S. Pat. No. 5,821,337 and 6,949,245). Chimeric anti-CD20 (U.S. Pat. No. 5,736,137) and two generic human IgG antibodies purchased from Sigma and from Jackson Laboratories, respectively were used as antibody controls. Prebleed sera, polysera obtained after immunization, and Media A were used as negative controls. Prebleed samples contained mouse sera prior to immunization, and polysera samples contained mouse anti-sera obtained after immunization. Media A (StemCell Technologies) was used for hybridoma clone growth and was a negative control in the ELISA.

ELISA data (Optical Density, O.D. 650 nm) were recorded for the binding responses of antibodies produced by hybridoma clones 10C4.1 and 2H9, and of the controls, and are shown below in Table 1.

TABLE 1

| | | anti- | anti- | | huIgG- | huIgG- |
|---|---|---|---|---|---|---|
| | anti-Her2^{a} | Her2^{b} | VEGF^{c} | anti-CD20^{d} | Sigma | Jackson |
| 10C4 | 0.848^{c} | 0.779 | 0.967 | 0.306 | 0.074 | 0.072 |
| 2H9 | 0.780 | 0.654 | 0.923 | 0.268 | 0.056 | 0.067 |
| polysera | 1.701 | 1.636 | 1.651 | 1.678 | 1.566 | 1.515 |
| prebleed | 0.182 | 0.118 | 0.085 | 0.306 | 0.059 | 0.058 |
| Media A | 0.172 | 0.106 | 0.088 | 0.272 | 0.065 | 0.065 |

Hybridoma clones 10C4.1 and 2H9.3.2 were identified as candidate pan-specific anti-hzAbAb displaying selective binding to the three humanized antibodies but not the chimeric antibody or generic IgG antibody. As shown in Table 1, the negative control samples, including prebleed sera and growth Media A, demonstrated low-level binding indicative of a generalized anti-human antibody binding response. As expected, polysera obtained post-immunization exhibited a binding response to each of the target humanized antibodies analyzed. The binding response to the chimeric antibody containing murine variable domains as similar to that of pre-bleed controls.

Clones 10C4.1 and 2H9.3.2 were further tested for binding responses to each of the seven humanized antibodies included in the antigen cocktail, as well as to humanized anti-BR3, a humanized antibody not present in the antigen cocktail. The humanized anti-BR3 antibody contains variable domain framework regions derived from a consensus sequence variable domain, as shown in FIGS. 2 and 3.

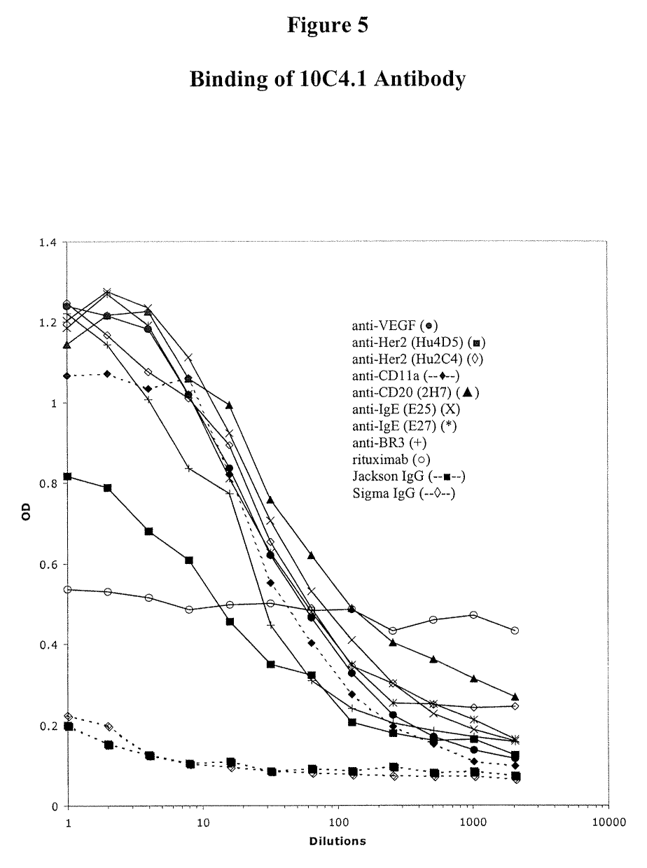

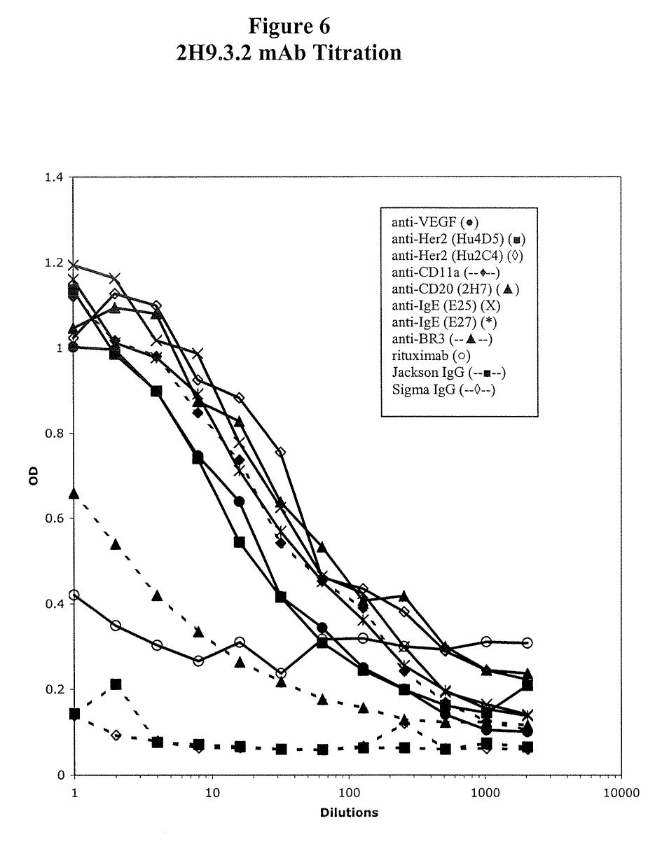

Hybridoma clones 10C4.1 and 2H9.3.2 were grown in Medium E (StemCell Technologies). Hybridoma supernatant was collected and purified over a mouse anti-human IgG column. The bound material was eluted with 1:10 dilution from 1 M glycine (pH 2.7) and used for titration analysis as shown in FIGS. 5 and 6.

Titration analysis was performed for the pan-specific anti-hzAbAb produced by each of the hybridoma clones, 10C4.1 and 2H9. Binding of the pan-specific antibodies at various dilutions was analyzed by ELISA and results were plotted against dilution, as shown in FIG. 5 (10C4) and FIG. 6 (2H9). Binding to each of the target humanized antibodies contained in the antigen cocktail, to the control chimeric antibody, and commercially available human IgG, and to an anti-BR3 antibody not contained in the antigen cocktail but having a related variable domain framework region, was analyzed.

FIG. 5 shows the binding of the anti-humanized antibody antibody (anti-hzAbAb) 10C4.1 to related humanized antibodies having variable domain framework regions sharing at least 80% sequence identity and also having at least 80% identity with framework regions of a variable domain consensus sequence derived from a Kabat compilation of antibody sequences. Data are shown for 10C4.1 antibody binding to anti-VEGF antibody (closed circles), anti-HER2 antibody (closed squares), anti-Her2 dimerization antibody (open diamonds), anti-CD 11a antibody (closed triangles), anti-IgE antibody (E25) (“X”), humanized anti-CD20 (closed triangles), anti-IgE antibody (E27) (asterisk), and anti-BR3 (+), chimeric anti-CD20 (rituximab) (open circles), and generic human IgGs from Jackson ImmunoResearch Laboratories (closed square with dashed line) and Sigma (open diamond with dashed line).

FIG. 6 shows the binding of the anti-humanized antibody antibody (anti-hzAbAb) 2H9.3.2 to related humanized antibodies having variable domain framework regions sharing at least 80% sequence identity and also having at least 80% identity with framework regions of a variable domain consensus sequence derived from a Kabat compilation of antibody sequences. Data are shown for 2H9.3.2 antibody binding to anti-VEGF antibody (closed circle), anti-Her2 antibody (Hu4D5) (closed square), anti-Her2 dimerization antibody (Hu2C4) (open diamond), anti-CD11a antibody (open diamond with dashed line), anti-IgE E25 antibody (“X”), anti-CD20 antibody (closed triangle), anti-IgE E27 antibody (asterisk), and anti-BR3 antibody (closed triangle with dashed line)). Chimeric anti-CD20, rituxamab (open circle), and generic human IgGs from Jackson ImmunoResearch Laboratories (closed square with dashed line) and Sigma (closed diamond with dashed line) were also included as negative controls. The optical density (OD) was measured at 650 nm. Dilutions (1:2) started at 10 μg/ml (=1 on the X-axis).

For the titration analysis, the initial concentration of 10C4.1 and 2H9.3.2 (with a dilution factor of 1) was 1 ug/ml. The optical density (OD) was measured at 650 nm. Dilutions (1:2) started at 10 μg/ml (=1 on the X-axis). In the) Figures, the x-axis represents subsequent dilution factors used to dilute the anti-hzAbAb clones in the assay.

As demonstrated in FIGS. 5 and 6, the two isolated hybridoma clones were confirmed as pan-specific anti-hzAbAb due to the demonstrated selective binding of antibodies produced from these hybridomas to a panel of related humanized antibodies and not to unrelated antibodies. Each hybridoma clone was deposited under the terms of the Budapest Treaty with the American Type Culture Collection, P.O. Box 1549 Manassas, Va. 20108:

Clone 10C4.1 deposited Jan. 13, 2006 and assigned ATCC No. PTA-7316.

Clone 2H9.3.2 deposited Jan. 13, 2006 and assigned ATCC No. PTA-7314.

ECLA Screening

ECLA was performed as described in Baker et al., 2002, Trends Biotechnol., 20:149-156. Briefly, separate batches of target humanized antibodies were labeled with biotin or Ori-tag (Igen International Inc, Gaithersburg, Md. and used as capture reagents). Humanized anti-CD20, anti-CD 11a, anti-VEGF, and generic human IgG (Jackson ImmunoResearch) were biotinlylated with biotinylamiocaproic acid-N-hydroxy-succinimid ester (Organics Inc.) using standard amine based chemistry and with ORI-TAG NHS ester according to the manufacturer's instructions.

Briefly, separate batches of antibodies were labeled with biotin or Ori-tag (Igen International Inc, Gaithersburg, Md.). Anti-CD20, anti-CD 11a, anti-VEGF, and Hu IgG (Jackson ImmunoResearch), were biotinlylated with biotinylamiocaproic acid-N-hydroxy-succinimid ester (Organics Inc.) using standard amine based chemistry at a target ratio of 2.5:1 biotin to anti-CD20 and 20:1 biotin to anti-CD11a, anti-VEGF, and Hu IgG. The molecules were labeled with ORI-TAG NHS ester according to the manufacturer's instructions at a targeted ratio of 2.5:1 ORI-TAG to anti-CD20 and 10:1 ORI-TAG to anti-CD 11a, anti-VEGF, and Hu IgG. A master working solution was prepared by mixing each biotinylated antigen and each Ori-tag labeled antigen in a 1:1 ratio. The final concentration of each labeled antigen in the master working solution was 0.5 μg/ml.

A volume of 50 μl of master working solution containing the capture reagent (specific target antibody antigen) and 50 μl of supernatant from individual hybridoma clones were added to a 96-well round-bottom polypropylene plate. Each well in the plate contained supernatant from a single hybridoma clone. The plate was incubated overnight at room temperature in the dark with a gentle agitation before the addition of 10 μg of streptavidin coated magnetic beads in a volume of 150 μl was added to each well. The plate was incubated for another one hour at room temperature in the dark with a gentle agitation. Post-incubation, the plates were read for electrochemiluminescence on an IGEN M384 (IGEN International, Inc.) analyzer using the following protocol parameters: bead type set at 2.80 microns, aspiration volume of 200 μl, POP of 0 mv, gain of 1, wash volume of 700 μl, clean cycle of 2, wash speed of 200 ml/sec. Data were collected and reported in electrochemiluminescence units (ECLU) (see Table 2). Medium A was used a control.

To be detected by ECLA, the analyte molecule must bind both a capture reagent (humanized antibody) and a detecting agent. For example, antibodies from the hybridoma supernatant culture that formed a complex with both the capture reagent and detecting agent were detected in the assay, (ECLA⁺). Antibodies from the hybridoma supernatant culture that bound only capture reagent or only detecting reagent were not detected. The negative control was hybridoma growth media, MEDIA A, measured at 266 ECLU. Both hybridoma clones 10C4.1 and 2H9.3.2 were detected in ECLA, and bound each of the humanized anti-CD20 and humanized anti-VEGF target antibodies. Neither clone 10C4.1 nor clone 2H9.3.2 bound generic human IgG from Jackson ImmunoResearch Laboratories. In fact, the binding response, measured in the ECLA assay, between both clones and the generic human IgG was less than the binding response between the clones and the negative control.

Shown below in Table 2, results from ECLA further confirmed the selective binding response of clones 10C4.1 and 2H9.3.2 to the related humanize antibodies.

TABLE 2¹

| Clone | Anti-CD20 | HuIgG-Jackson | Anti-VEGF |
|---|---|---|---|
| 10C4 | 2127 | 235 | 4246 |
| 2H9 | 1120 | 237 | 2733 |

¹Units in ECLU.

Biacore Analysis

Hybridoma clones 2H9.3.2 and 10C4.1 were further characterized and confirmed in Biacore assays for pan-specific anti-hzAbAb binding response by determining dissociation equilibrium constants (K_{D}) for binding. Hyridoma clones 2H9.3.2 and 10C4.1 were grown in Medium E (StemCell Technologies). Hybridoma supernatant was collected and purified over a mouse anti-human IgG column. The bound material was eluted with 1: 10 dilution from 1 M glycine (pH 2.7) and used for titration analysis as shown in FIGS. 5 and 6.

The binding responses for hybridoma clones 2H9.3.2 and 10C4.1 were calculated generally as described in (Lofas & Johnson, 1990, J. Chem. Soc. Commun. 21, 1526-1528), from association and dissociation rate constants measured in a BIAcore™-3000 (Biacore AB, Piscataway, N.J.) surface plasmon resonance system.

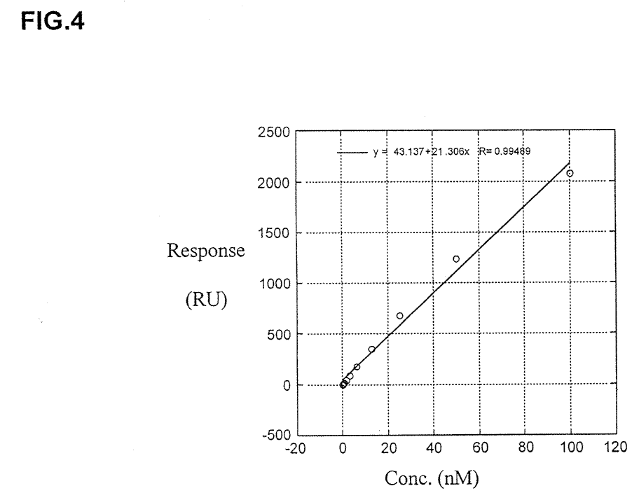

To determine the concentration of monoclonal antibodies in the supernatant from the individual hybridoma clones, a standard curve was generated. The standard curve utilized purified polyclonal antibodies raised against an anti-humanized VEGF antibody. This concentration curve is shown at FIG. 4, and was used to calculate dissociation rates constants (K_{dissoc}) association rate constants (K_{assoc}) and dissociation equilibrium constants (K_{D}) (Table 3). The equilibrium constants and rate constants were obtained using BIA evaluation 3.2 software (Biacore AB, Piscataway, N.J.) provided by the manufacturer. Dissociation equilibrium constants were obtained using the ratio of K_{assoc}/K_{dissoc}. The fitting model assumed 1:1 binding.

To determine the concentration of a monoclonal antibody in the hybridoma supernatant, a biosensor chip was activated for covalent coupling of humanized anti-VEGF antibody using N-ethyl-N′-(3-dimethylaminopropyl)-carbodiimide hydrochloride (EDC) and N-hydroxysuccinimide (NNS) according to the manufacturer's (BlAcore) instructions. Humanized anti-VEGF antibody was diluted into 10 nM sodium acetate buffer (pH 5.0) to approximately 30 ug/ml and injected over the chip to obtain a signal of 6928 in response units (RU) of immobilized material. Finally, 1M ethanolamine was injected as a blocking agent. Regenerations were carried out with 10 mM Glycine, pH 2.0. An in-line reference flow cell was created using the same activation and deactivation methods as described above, but with no proteins immobilized. Serial dilutions of purified anti-humanized anti-VEGF antibody in HBS-EP buffer (0.01 M HEPES, pH 7.4, 0.15 M NaCl, 3 mM EDTA, 0.005% surfactant P20) were injected over both the reference and humanized anti-VEGF antibody flow cells. After subtracting reference flow cell, the binding response in Relative Unit (RU) for each anti-humanized anti-VEGF antibody dilution was plotted against the concentration (FIG. 4). A linear fitting of the standard curve was performed and was used to calculate the concentration of 10C4.1 and 2H9.3.2 in the supernatants.

For kinetics measurements, humanized anti-VEGF antibody was immobilized to another flow cell with a response of 561 relative unit (RU). Two-fold serial dilutions of antibodies were injected over the sensor chip in HBS-EP buffer (0.01 M HEPES, pH 7.4, 0.15 M NaCl, 3 mM EDTA, 0.005% surfactant P20) at 25° C. using a flow rate of 30 ul/min.

TABLE 3

| | | | | Estimated concentration |
|---|---|---|---|---|
| Clone | K_{assoc} (1/Ms) | K_{dissoc} (1/s) | K_{D} (M) | in the supernatant* (nM) |
| 2H9 | 2.41E+02 | 3.03E−04 | 1.26E−08 | 13.9 |
| 10C4 | 1.25E+05 | 1.66E−04 | 1.33E−09 | 84.5 |

Concentrations were estimated using a standard curve generated with purified polyclonal anti-humanized anti-VEGF antibodies with Biacore (see FIG. 3).

Further Biacore analysis was performed to determine if 10C4.1 bound to a generic human IgG. Biacore responses for 10C4.1 against immobilized humanized anti-VEGF antibodies and commercially available human IgG (Jackson ImmunoResearch) were measured and are shown in Table 4.

TABLE 4

| | 10C4.1 Binding | | |
|---|---|---|---|
| Capture Reagent | 80 nM (ru) | 40 nM (ru) | 20 nM (ru) |
| monoclonal anti-VEGF | 1435.7 | 1060.7 | 646.1 |
| Hu IgG | 8.3 | 4.6 | 1.6 |

Briefly, both anti-VEGF and human IgG (Jackson ImmunoResearch) antibodies were immobilized on a CM5 sensor chip (Biacore, Inc., Piscataway, N.J.) at similar levels (6540 RU and 6928 RU for huIgG and anti-VEGF humanized antibody, respectively) and various concentrations of 10C4.1 supernatant (as determined by standard curve in FIG. 4) were allowed to flow through. Responses were recorded 20 seconds after the end of the sample injection.

The results demonstrate that clones 10C4.1 and 2H9.3.2 selectively bound to members of the related group of humanized antibodies in the antigen cocktail. Since each humanized antibody of the antigen cocktail had a different set of CDRs, the pan-specific anti-hzAbAb clones are presumed to bind the variable domain framework regions.

The method described herein can be generalized to other anti-framework antibody productions and applications.

### Example 3

### Applications of a Pan-Specific Anti-Humanized Framework Region Antibody

Anti-hzAbAb generated against related variable domain framework regions, for example, having framework regions sharing at least 80% identity with the framework regions of a variable domain consensus sequence can be generated by methods described herein. Such antibodies can be used in generic pharmacokinetic (PK) assays to measure drug concentration in preclinical studies of a monoclonal antibody therapeutic that uses the same Kabat-defined consensus framework region.

The PK parameters for a 24-hour plasma concentration versus time profile for a new humanized antibody with the consensus framework can be calculated according to a one-compartment model (first order absorption and elimination kinetics) using standard methods (Winningly Professional 3.2, Parasite Corp., Mountain View, Calif.). The plasma concentration versus time data fits to a two-exponential model, one exponential for absorption and one for elimination, and the PK parameters are calculated from the slopes and intercept, for example. It is generally assumed that there is no absorption lag time. Parameters calculated can include the apparent absorption and terminal half-life, distribution volume (V/F), and clearance (CL/F). The distribution volume and clearance generally are not corrected for bioavailability (F). Cₘₐₓ and Tₘₐₓ represent the maximal concentration and the time when maximal IF-I concentration were observed, respectively, as fitted by the model. The concentration of a new humanized antibody at 0 time is generally subtracted prior to performing the PK analysis.

### REFERENCES

References cited within this application, including patents, published applications and other publications, are hereby incorporated by reference.

1. A process for producing a pan-specific antibody or antigen binding fragment thereof comprising:
a) immunizing an animal with a plurality of related antibodies, or fragments thereof, to produce anti-antibody antibodies, wherein said related antibodies, or fragments thereof, comprise variable domain framework regions that share at least 80% amino acid sequence identity; and
b) selecting from the produced anti-antibody antibodies a pan-specific antibody, or antigen binding fragment thereof, that selectively binds two or more related antibodies, or antigen binding fragments thereof, wherein the at least two related antibodies, or antigen binding fragments thereof, comprise variable domain framework regions that share at least 80% sequence identity.

2. The process of claim 1, wherein the variable domain framework regions share at least 85%, 90%, or 95% amino acid sequence identity.

3. The process of claim 1, wherein the animal is immunized with at least two antibodies, or fragments thereof, that bind different antigens.

4. The process of claim 1, wherein the animal is immunized with 3, 4, 5, 6, 7, 8, 9, or 10 antibodies, or fragments thereof, that bind different antigens.

5. The process of claim 1, wherein said selected pan-specific antibody, or antigen binding fragment thereof, selectively binds two or more related antibodies, or fragments thereof, that bind different antigens.

6. The process of claim 1, wherein the animal is immunized with two or more antibodies, or fragments thereof, each comprising a variable domain framework region having at least 80% amino acid sequence identity with the framework region of a variable domain consensus sequence.

7. The process of claim 6, wherein the variable domain framework regions of the two or more antibodies, or fragments thereof, share at least 85%, 90%, or 95% amino acid sequence identity with the framework region of a variable domain consensus sequence.

8. The process of claim 1, wherein said selecting is by one or more of ELISA, ECLA, and Biacore analysis.

9. A process for producing a pan-specific antibody or antigen binding fragment thereof comprising:
a) immunizing an animal with a plurality of related antibodies, or fragments thereof, to produce anti-antibody antibodies, wherein said related antibodies, or fragments thereof, each comprise variable domain framework regions that share at least 80% amino acid sequence identity with framework regions of a variable domain consensus sequence; and
b) selecting from the produced anti-antibody antibodies a pan-specific antibody, or antigen binding fragment thereof, that selectively binds two or more related antibodies, or fragments thereof, wherein the at least two related antibodies, or fragments thereof, each comprise variable domain framework regions that share at least 80% amino acid sequence identity with framework regions of a variable domain consensus sequence.

10. The process of claim 9, wherein the variable domain framework regions of the plurality of related antibodies, or fragments thereof, share at least 85%, 90%, or 95% amino acid sequence identity with the framework regions of a variable domain consensus sequence.

11. The process of claim 9, wherein the variable domain consensus sequence comprises at each position an amino acid that is most abundant for a corresponding residue in a compilation of amino acids present in a class or subclass of human antibodies.

12. The process of claim 11, wherein the consensus sequence comprises at each position an amino acid residue identified as most abundant at that position in a Kabat compilation of human antibody variable domain sequences.

13. The process of claim 11, wherein the variable domain consensus sequence comprises a heavy chain variable domain Subgroup III consensus sequence, a light chain variable domain kappa Subgroup I consensus sequence, or both.

14. The process of claim 1, wherein the animal is immunized with one or more antibody, or fragment thereof, that comprises a variable domain consensus sequence framework region having 1, 2, 3, 4, 5, 6, 7, 8, 9, or 10 amino acid substitutions.

15. The process of claim 1, wherein the animal is immunized with two or more humanized antibodies, or fragments thereof, that bind an antigen selected from: Her2, VEGF, IgE, CD11a, and CD20.

16. The process of claim 1, wherein the animal is immunized with 3, 4, 5, 6, or 7 humanized antibodies that bind one or more antigen selected from: Her2, VEGF, IgE, CD11a, and CD20.

17. The process of claim 1, wherein said immunizing is with two or more antibodies, or fragments thereof, that comprise any of the framework regions shown in FIG. 2 or FIG. 3.

18. A pan-specific anti-humanized antibody antibody (anti-hzAbAb) produced by the process claim 1.

19. A method for detecting a pan-specific anti-humanized antibody antibody (anti-hzAbAb) comprising:
a) analyzing an antibody sample for selective binding of an anti-humanized antibody to at least two humanized antibodies, or fragments thereof, comprising variable domain framework regions that share at least 80% sequence identity; and
b) identifying as a pan-specific anti-hzAbAb an antibody that selectively binds said two or more humanized antibodies, or fragments thereof.

20. An article of manufacture comprising: a container, a composition within the container comprising the pan-specific antibody of claim 18, and a package insert containing instructions for use of the composition.

21. An immunoassay kit comprising the pan-specific antibody of claim 17 and assay reagents.

22. A method for detecting a humanized antibody, or antigen binding fragment thereof, in an antibody sample, the method comprising:
a) contacting a sample with the pan-specific antibody of claim 18; and
b) detecting a humanized antibody, or antigen binding fragment thereof, that binds the pan-specific antibody.

23. The method of claim 21, wherein said detected humanized antibody, or antigen binding fragment thereof, comprises a variable domain framework region having at least 80% sequence identity with framework regions of a variable domain consensus sequence.

24. The method of claim 21, wherein said detected humanized antibody, or antigen binding fragment, thereof comprises a variable domain framework region having at least 85%, 90%, or 95% amino acid sequence identity with the framework region of a variable domain consensus sequence.

25. The method of claim 22, wherein the variable domain consensus sequence comprises at each position an amino acid that is most abundant for a corresponding position in a compilation of amino acids present in a class or subclass of human antibodies.

26. The method of claim 22, wherein the consensus sequence comprises at each position an amino acid residue identified as most abundant at that position in a Kabat compilation of human antibody variable domain sequences.

27. The method of claim 21, wherein the detected humanized antibody comprises a variable domain consensus sequence framework region having 1, 2, 3, 4, 5, 6, 7, 8, 9, or 10 amino acid substitutions.

28. The method of claim 21, wherein the consensus sequence comprises a heavy chain variable domain Subgroup III consensus sequence, a light chain variable domain kappa Subgroup I consensus sequence, or both.

29. The method of claim 21, wherein the detected humanized antibody binds an antigen selected from: Her2, VEGF, IgE, CD11a, and CD20.

30. The pan-specific anti-humanized antibody antibody (anti-hzAbAb) of claim 18, comprising an antibody produced from hybridoma clone 10C4.1, having ATCC Accession No. PTA-7316.

31. The pan-specific anti-humanized antibody antibody (anti-hzAbAb) of claim 18, comprising an antibody produced from hybridoma clone 2H9.3.2, having ATCC Accession No. PTA-7314.
